(19) United States
(12) Patent Application Publication
(10) Pub. No.: US 2015/0139973 A1
(43) Pub. Date: May 21, 2015

(54) OPHTHALMOLOGICAL VEHICLE SYSTEM FOR DRUGS, OPHTHALMOLOGICAL KIT AND ALSO USE OF AN OPHTHALMOLOGICAL COMPOSITION

(71) Applicant: F. HOLZER GMBH, St. Ingbert (DE)

(72) Inventors: Ute Steinfeld, St. Ingbert (DE); Frank Holzer, St. Ingbert (DE); Hyeck Hee Lee, St. Ingbert (DE); Markus Mahler, Volklingen (DE)

(21) Appl. No.: 14/401,464

(22) PCT Filed: May 14, 2013
(86) PCT No.: PCT/EP2013/059927

§ 371 Date: Nov. 14, 2014

(30) Foreign Application Priority Data
May 15, 2012 (EP) 12168139.9

(51) Int. Cl. A61K 9/00; A61K 31/355; A61K 31/375; A61K 31/197; A61K 31/05; A61F 9/00; A61K 47/14; A61K 47/24; A61K 47/12; A61K 47/44; A61K 31/573; A61K 31/122; A61K 45/06

(52) U.S. Cl. CPC A61K 9/0048; A61K 31/122; A61K 31/355; A61K 31/375; A61K 31/197; A61K 31/05; A61K 45/06; A61K 47/14; A61K 47/24; A61K 47/12; A61K 47/44; A61K 31/573; A61F 9/0008
USPC 424/94.4; 424/94.1; 514/689; 514/458; 514/474; 514/561; 514/179; 514/171; 514/2.9; 514/401; 514/324; 514/235.5; 514/165; 604/298

(57) ABSTRACT

The present invention relates to an ophthalmological vehicle system for the permeation and/or for the active substance transport of ophthalmological active substances through the cornea and/or the sclera of the eye of mammals. This vehicle system assists the transport of the active substances through the cornea and/or the sclerotic tissue of the eye. The vehicle system is suitable for the prophylaxis and/or treatment of diseases of the front and/or back portion of the eye. Likewise, the present invention relates to an ophthalmological kit, comprising a special ophthalmological composition and also, as separate formulation, an ophthalmological active substance. In addition, the use of a special ophthalmological composition as vehicle system, penetration accelerator, penetration enhancer, absorption enhancer/-improver/-accelerator for the permeation and/or for the active substance transport of ophthalmological active substances through the cornea and/or the sclera of the eye of mammals is the subject of the invention. Furthermore, the invention relates to a fluid dispenser which comprises an ophthalmological vehicle system according to the invention.

The present invention relates to an ophthalmological vehicle system for the permeation and/or for the active substance transport of ophthalmological active substances through the cornea and/or the sclera of the eye of mammals. This vehicle system assists the transport of active substances through the cornea and/or the sclerotic tissue of the eye. The vehicle system is suitable for the prophylaxis and/or treatment of diseases of the front and/or back portion of the eye. Likewise, the present invention relates to an ophthalmological kit, comprising a special ophthalmological composition and also, as separate formulation, an ophthalmological active substance. In addition, the use of a special ophthalmological composition as vehicle system, penetration accelerator, penetration enhancer, absorption enhancer/-improver/-accelerator for the permeation and/or for the active substance transport of ophthalmological active substances through the cornea and/or the sclera of the eye of mammals is the subject of the invention. Furthermore, the invention relates to a fluid dispenser which comprises an ophthalmological vehicle system according to the invention.

Active substances applied topically on the cornea often do not reach the eye interior, or not in therapeutic concentrations, i.e. the front or back eye portion. The bioavailability, i.e. the effective transport of the active substance through the cornea or sclera into the aqueous humour, respectively into the vitreous body, to the active site, is influenced by various factors:
the physicochemical properties of the active substance and
the permeability of the anatomical barrier
precorneal factors such as tear secretion or nasolachrymal discharge.

The lipophilic epithelium of the cornea thereby forms the main barrier for hydrophilic active substances because the Zonulae occludentes with the tight junctions surround the surface cells of the epithelium such that the paracellular cavity is quasi-sealed. Hence the tight junctions limit the permeation of molecules between the cells.

A corresponding lipophilicity of the drugs is a precondition for transcellular permeation in the corneal epithelium, both the lipophilic cell membrane and the hydrophilic plasma thereby also requiring to be passed through.

WO 2012/059158 A1 describes a composition which comprises at least one ω-3 fatty acid and also at least one modulator, e.g. an inhibitor, antagonist etc., of the NF-B transcription factor. This composition is suitable as drug or pharmaceutical base formulation, in particular for the prophylaxis or treatment of inflammations. In the case of this composition, a sustained release of the contained ω-3 fatty acids is known.

From WO 95/05163 A1, a water-based lipid composition is known, which has an adhering effect, e.g. on the cornea of the eye, so that active substances once applied remain longer at the active site and hence an extension of the effect can be achieved.

In order to improve the penetration of the active substance, frequently quaternary ammonium compounds, such as benzalkonium chloride, are used in eye drops and act simultaneously also as preservative. It has been proved that benzalkonium chloride attacks and damages the cornea of the eye as far as the deeper cell layers. Also other penetration enhancers, such as DMSO, sodium glycocholate, sodium fusidate, etc. have a damaging effect.

Hence it was the object of the present invention to indicate a composition which enables effective active substance transport to the eye whilst avoiding the above-mentioned problems.

This object is achieved, with respect to an ophthalmological vehicle system, with the features of patent claim 1, with respect to an ophthalmological kit, with the features of patent claim 16 and also with respect to possibilities of use of an ophthalmological composition, with the features of patent claim 17. A fluid dispenser, comprising an ophthalmological vehicle system according to the invention is indicated by patent claim 18. The dependent patent claims thereby represent advantageous developments.

According to the invention, an ophthalmological vehicle system for the permeation and/or for the active substance transport of ophthalmological active substances through the cornea and/or the sclera of the eye of mammals in the prophylaxis and/or treatment of diseases of the front and/or back portion of the eye is provided, comprising:

a) ≧30 to 99.95% by weight, relative to the total composition, of at least one fatty acid ester,

b) 0.01% by weight to ≦50% by weight, relative to the total composition, of one or at least one emulsifier, and also

c) at least one ophthalmological active substance, selected from the group consisting of antibiotics, corticoids, local anaesthetics, decongestants, non-steroidal antiphlogistics, virustatics, antiseptics, cortisone, anti-allergic active substances, prostaglandin analogues, active substances from the active substance class of antihistamines and/or corticosteroids, anti-allergic active substances, pantothenic acid derivatives, non-steroidal anti-inflammatory drugs, vascoconstrictors and/or anti-glaucoma active substances in a pharmaceutically effective concentration.

The eye can be subdivided into two portions: the back and the front eye portion. Both portions are separated from each other spatially by lens and iris. The terms front and back eye portion are defined as follows according to the invention:

front eye portion: comprises sclera, cornea, anterior eye chamber, iris and lens.

back eye portion: comprises ciliary body, vitreous body, retina, optic papilla, choroid and sclera.

The present invention describes a formulation which reversibly reduces the barrier function of the cornea and thus increases or accelerates the effective permeation and penetration of an active substance through the cornea. It is of great importance that the barrier function of the cornea is rapidly regenerated again and not irreversibly destroyed.

The lipophilic base of the formulation offers, relative to aqueous formulations, the advantage that it has a substantially longer dwell time in the precorneal region and hence releases active substance into the eye over a longer time.

A further advantage is the viscoelastic property of the formulation, i.e. between blinks it is more viscous (higher-viscous), during the blink thinner (lower-viscous). Hence it forms a gel-like protective film on the eye surface which becomes thinner when blinking, feels pleasant and does not impede vision.

In contrast to most lipophilic formulations, such as ointments and oils, which cause blurred vision due to their high refractive index, the refractive index of the present formulation at approx. 1.43 (according to the precise composition) is in the range recommended for eye preparations (Siebenbrodt and Keipert, 1991).

Furthermore, the formulation is non-irritating and is distinguished by excellent tolerability, which is of particular importance for ophthalmic agents.

A preferred embodiment provides that the fatty acid ester is selected from the group consisting of isopropyl myristate and isopropyl palmitate.

Furthermore, it is advantageous if the emulsifier represents a lecithin or a lecithin-containing composition, preferably is selected from phosphatidylcholine-containing compositions with a phosphatidylcholine content of at least 90% by weight, further preferred at least 95% by weight, in particular Epikuron 200 (phosphatidylcholine with ≧98% by weight purity) or Epikuron 100.

In addition or alternatively to lecithins, likewise emulsifiers are suitable with HLB values of 2-7, in particular ethoxylated triglycerides, such as PEG-5 castor oil (HLB=3.9), PEG-6 diricinoleates (HLB=5.0), PEG-7 hydrogenated castor oil (Cremophor® WO 7, HLB=5.0); sorbitan esters such as sorbitan oleates (Span® 80, HLB=4.5), sorbitan stearates (HLB=5.0), sorbitan sesquioleates (Crill® 43=HLB=3.7), sorbitan isostearates (Crill® 6, HLB=4.7), sorbitan tristearates (Crill®35, HLB=2.1); polyethoxylated fatty acids and—alcohols such as PEG-2 oleates (HLB=5.0), PEG-4 distearates (HLB=3.0); PEG-2 stearates (HLB=4.4), ceteareth-3, (Volpo® CS3, HLB=5.0), ceteth-2 (Volpo® C2, HLB=5.3); and also mixtures hereof.

A particularly preferred embodiment of the present invention provides that merely a single emulsifier is contained in the ophthalmological composition, in particular phosphatidylcholine (Epikuron 200).

A further preferred embodiment provides that the total content of the at least one fatty acid ester, relative to the total composition, is from 50 to 99.9% by weight, preferably 70 to 99.5% by weight.

Likewise it is preferred if the total content of the one or at least one emulsifier, relative to the total composition, is from 0.05 to 15% by weight, preferably from 0.1 to 15% by weight, further preferred from 0.5 to 12% by weight, further preferred from 1 to 10% by weight, particularly preferred from 2 to 8% by weight, in particular from 5 to 7% by weight.

In addition, the ophthalmological vehicle system according to the present invention can comprise at least one ω-3 fatty acid and/or one ω-3 fatty acid derivative selected from the group consisting of esters, mono-, di- or trigylcerides, lipids, oxygenation products hereof, such as e.g. alcohols, aldehydes, ketones, epoxides etc., carboxylate salts, amides, other pharmacologically acceptable carboxylic acid derivatives and mixtures hereof. Examples of such omega-3 fatty acid derivatives are the resolvins (e.g. of series E and D, such as e.g. resolvin E1 (RvE1) and resolvin E2 (RvE2) and also the isomers thereof 18S-RvE1 and 18S-RvE2), protectins (e.g. of series E and D, such as e.g. protectin D1 and D2), neuroprostanes, such as e.g. A4-NP, isoprostanes or the synthetic analogues thereof.

The at least one ω-3 fatty acid can thereby be contained as free acid, but also be present in derivatised, i.e. modified form. There should be understood as ω-3 fatty acid derivatives in the sense of the present invention, all compounds which are derived from a ω-3 fatty acid or comprise a ω-3 fatty acid as structural element. Hence, there are included therein for example salts of ω-3 fatty acids, substances which have a covalently bonded ω-3 fatty acid or substance mixtures which comprise a ω-3 fatty acid as integral component.

ω-3 fatty acids are multiply unsaturated fatty acids and belong to the essential fatty acids which are ingested as a rule with food and are incorporated in the body in cell membranes.

The secondary products of these fatty acids are tissue hormones and act as substances which are intrinsic to the body and effective in a regulatory manner. They influence numerous metabolic processes and functions.

As a function of special stimuli, e.g. due to neutral irritations or other mediators, e.g. histamines, ω-3- and ω-6 fatty acids are released from the membrane lipids and made available for the biosynthesis of these tissue hormones, the eicosanoids.

These act in fact in very low concentrations (between 10⁻⁸ and 10⁻¹⁰ mol per litre) as mediators directly at the site of their production. The effects are exerted either by a paracrinal route at adjacent cells or by an autocrinal route at the producing cell itself. The range of these mediators is limited by their very short lifespan of seconds up to a few minutes. Thus, even with topical application or inhalation, there is a favourable effect on the composition and a locally limited effect is achieved, which is advantageous for therapeutic application. In addition, the fatty acid composition also has an effect on the permeability and fluidity of the membranes.

It is hereby preferred if the at least one ω-3 fatty acid is selected from the group consisting of α-linolenic acid, stearidonic acid, eicosatetraenoic acid, eicosapentaenoic acid (EPA), docosapentaenoic acid (DPA), docosahexaenoic acid, resolvins, hexadecatrienoic acid, eicosatrienoic acid, heneicosapentaenoic acid, tetracosapentaenoic acid, tetracosahexaenoic acid, oxygenation products derived herefrom, such as e.g. alcohols, aldehydes, ketones, epoxides etc. and also mixtures and combinations hereof.

Likewise, it is possible that the at least one ω-3 fatty acid is contained in the form of an ester of an organic alcohol, preferably of a linear or branched aliphatic monovalent alcohol with 1 to 18 carbon atoms, particularly preferred as methyl-, ethyl-, n-propyl-, i-propyl-, n-butyl-, i-butyl-, t-butyl ester.

Alternatively hereto, it is likewise conceivable that the at least one ω-3 fatty acid is contained in the form of a plant or animal oil which comprises, in addition to the at least one ω-3 fatty acid, also at least one ω-6 fatty acid, the molar ratio of the ω-3 fatty acid to the ω-6 fatty acid being from 100:1 to 1:100, preferably 20:1 to 1:10, further preferred 15:1 to 1:1, particularly preferred 8:1 to 2:1.

Furthermore it is advantageous if the at least one ω-3 fatty acid is present in the form of an oil, selected from the group consisting of algal oil, fish oil, perilla oil, shi oil, linseed oil, cameline oil, Sacha Inchi oil, rapeseed oil, olive oil, evening primrose oil, soya oil, hemp oil, walnut oil, peanut oil, sesame oil, maize oil, flaxseed oil and/or mixtures hereof.

Likewise, also combination possibilities of esters and oils of the at least one ω-3 fatty acid are conceivable.

In the case where ω-3 fatty acids are contained in the ophthalmological vehicle system, it is of advantage if the content of the at least one ω-3 fatty acid and/or of the derivative, relative to the total composition, is between 0.01 and 49.9% by weight, preferably between 0.05 and 30% by weight, particularly preferred between 0.05 and 10% by weight.

Further advantages are produced if the composition is free of compounds, selected from the group consisting of quaternary ammonium compounds, in particular benzalkonium chloride; sodium glycocholate and/or sodium fusidate. It is likewise advantageous if the composition is kept free of the following normal ointment bases: paraffins, lanolins, vaseline etc.

It is conceivable that merely a single ophthalmological active substance is contained, however likewise combination preparations are possible.

The at least one ophthalmological active substance is contained in this case preferably at 0.01 to 40% by weight, further preferred at 0.05 to 20% by weight, in particular from 0.1 to 10% by weight, relative to the total vehicle system. The type, the number and also the exact content of the at least one active substance respectively can thereby be adapted specifically to the respective application field of the vehicle system.

For individual selected active substances, the following preferred concentration ranges thereby apply:

For antibiotics, virostatics, corticoids, cortisone:

At least one ophthalmic active substance, preferably at 0.01-10% by weight, further preferred at 0.05-5% by weight, in particular from 0.1-3% by weight, relative to the total vehicle system.

Anti-allergic agents, non-steroidal anti-inflammatory drugs:

At least one ophthalmic active substance preferably at 0.01-5% by weight, further preferred at 0.05-3% by weight, in particular from 0.05-2% by weight, relative to the total vehicle system.

The active substances can thereby be selected from natural, synthetic or biotechnologically produced active substances. Special examples thereof are indicated subsequently:

Antibiotics:
polypeptide antibiotics: bacitracin, polymyxin B, gramicidin,
aminoglycosides: neomycin, framycetin, gentamicin, tobramycin,
sulphonamides: sulfacetamide
quinolones: ciprofloxacin, ofloxacin, lomefloxacin, moxifloxacin,
other antibiotics: chloramphenicol, fusidic acid

Alternatively, alone or in combination with ocular glucocorticoids
decongestants, such as naphazoline, phenylephrine, tetryzoline, tramazoline, xylometazoline;
non-steroidal antiphlogistics, such as diclofenac, indometacin;
virustatics, such as aciclovir;
antiseptics, such as cortisone, such as hydrocortisone, rimexolon;
anti-allergic active substances from antihistamines, corticosteroids, synthetic mast cell degranulation inhibitors and leukotriene receptor antagonists;
prostaglandin analogues, antibiotics;
at least one active substance from the active substance class of antihistamines,
and/or at least one active substance from the active substance class of corticosteroids;
the group of antihistamines ketotifen, thonzylamine, mepyramine, thenalidin, tripelenamine, chloropyramine, promethazine, tolpropamin, dimetinden, clemastin, bamipine, isothipendyl, diphenhydramine, diphenhydramine methylbromide, chlorophenoxamine, pheniramine, diphenylpyraline, dioxopromethazine, dimenhydrinate, thiethylperazine and meclozine, azelastine, levocabastine, astemizole, mebhydroline, terfenadine, mequitazine, cetirizin, emedastin, mizolastin, olopatadine, epinastine and antazolin;
the group of corticosteroids triamcinolone, dexamethasone, hydrocortisone, hydrocortisone acetate, hydrocortisone butyrate, hydrocortisone buteprat, prednisolone, betamethasone, methylprednisolone, clobetasone, flumetasone, fluocortin, fluperolon, fluorometholone, flupredniden, desonide, triamcinolon, alclometason, dexamethasone, clocortolone, betamethasone, fluclorolon, desoximetasone, fluocinolonacetonide, fluocortolone, diflucortolone, fludroxycortide, fluocinonid, budesonid, diflorason, amcinonid, halometason, mometason, methylprednisolonaceponate, beclo-metason, hydrocortisone aceponate, fluticason, prednicarbate, prednisone, prednisolone, difluprednate, ulobetasol, clobetasol, halcinonide, medryson, desonid, formocortal, rimexolon, mazipredon, flunisolid and tixocortol;
at least one anti-allergic active substance from the group cromoglicic acid, spaglumic acid, lodoxamide, nedocromil, montelukast and zafirlukast;
pantothenic acid derivatives dexpanthenol, DL-panthenol, salts of pantothenic acid (e.g. Na-pantothenate, Ca-pantothenate), esters of pantothenic acid (e.g. ethyl-, methylester), panthenol ether (e.g. ethyl- or methylether), panthenol thioether and panthenyl triacetate, particularly preferred dexpanthenol (=D-(+)-pantothenyl alcohol);
alternatively non-steroidal anti-inflammatory drugs (“NSAIDs”), such as e.g.

aminoarylcarboxylic acid derivatives (e.g. enfenamic acid, etofenamate, flufenamic acid, isonixine, meclofenaminic acid, mefenaminic acid, nifluminic acid, talniflumate, terofenamate, tolfenamic acid), arylacetal acid derivatives (e.g. aceclofenac, acemetacin, alclofenac, amfenac, amtolmetinguacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, ionazolac, metiazic acid, mofezolac, oxametacin, pirazolac, proglumetacin, sulindac, tiaramide, tolmetin, tropesin, zomepirac), arylbutyric acid derivatives (e.g. bumadizon, butibufen, fenbufen, xenbucin), arylcarboxylic acid (e.g. clidanac, ketorolac, tinoridine), arylpropionic acid derivatives (e.g. alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirofen, pranoprofen, protizinic acid, suprofen, tiaprofenic acid, ximoprofen, zaltoprofen), pyrazoles (e.g. difenamizol, epirizol), pyrazolones (e.g. apazone, benzpiperylone, feprazone, mofebutazone, morazone, oxyphenbutazone, phenylbutazone, pipebuzone, propyphenazone, ramifenazone, suxibuzone, thiazolinobutazone), salicylic acid derivatives (e.g. acetaminosalol, aspirin, benorylate, bromosaligenin, calcium acetylsalicylate, diflunisal, etersalate, fendosal, gentisic acid, glycol salicylate, imidazolsalicylate, lysin acetylsalicylate, mesalamine, morpholine salicylate, 1-naphthyl salicylate, olsalazine, parsalmide, phenylacetyl salicylate, phenyl salicylate, salacetamide, salicylamide o-acetic acid, salicylsulphonic acid, salsalate, sulphasalazine), thiazincarboxamides (e.g. ampiroxicam, droxicam, isoxicam, lornoxicam, piroxicam, tenoxicam), 8-acetamidecapric acid, S(5′-adenosyl)-L-methionine, 3-amino-4-hydroxybutyric acid, amixetrine, bendazac, benzydamine, α-bisabolol, bucolome, difenpiramide, ditazol, emorfazon, fepradinol, guaiazulene, nabumetone, nimesulide, oxaceprol, paranyline, perisoxal, proquazone, superoxide-dismutase, tenidap, zileulon, the physiologically acceptable salts thereof and also combinations and mixtures hereof.

Other non-steroidal anti-inflammatory drugs (“NSAIDs”), which in addition can be contained in the composition according to the invention, comprise cyclooxygenase inhibitors, e.g. selective inhibitors of cyclooxygenase of type II, such as e.g. celecoxib and etodolac, PAF (platelet activating factor) antagonists, such as for instance apafant, bepafant, minopafant, nupafant, and modipafant; PDE (phosphodiesterase)-IV inhibitors, such as for instance ariflo, torbafylline, rolipram, filaminast, piclamilast, cipamfylline and roflumilast; inhibitors of cytokine formation, such as for instance inhibitors of the NF-κB transcription factor; or other known anti-inflammatory agents.

The pharmaceutical composition according to the invention can comprise, from the group of vasoconstrictors, e.g. oxymetazoline, xylometazoline, tretryzoline, naphazoline, tramazoline and/or the derivatives thereof as active substance component.

The composition according to the invention can comprise furthermore active substances with antiangiogenic effect, e.g. VEGF inhibitors e.g. VEGF aptamers or antibodies, as drug for the treatment of age-dependent macular degeneration (AMD), e.g. macugen, lucentis, avastin inter alfa.

In addition to the fatty acids, possibly also anti-glaucoma active substances can be added, such as
beta-blockers: timolol, levobunolol,
cholinergics: carbachol, pilocarpine,
alpha-2-adrenoreceptors-agonist: clonidine, brimonidine,
carboanhydrase inhibitors: brinzolamide, dorzolamide and acetazolamide,
prostaglandins: latanoprost, travoprost, bimatoprost, tafluprost, in order to influence the effect even more.

The concentration of the alternatively added agents which are contained in the present invention can vary according to the agent and type of disease. The concentration should suffice to treat for example an inflammation in the treated tissue or to prevent this. Typically, the concentrations are thereby in the range of 0.0001 to approx. 5% wt/wt (or alternatively at 0.01 to approx. 2% wt/wt, or from approx. 0.05% to 1%, or from approx. 0.01% to approx. 0.5% wt/wt).

Likewise, antibodies, aptamers, siRNA or other “small molecules” can be contained in order to prevent or treat diseases of the eye.

Likewise, in addition modulators, e.g. inhibitors, antagonists, in particular modulators of the NF-κB transcription factor, can be contained.

Immunological processes, processes in inflammatory courses and wound-healing processes form a tightly interwoven interaction in the case of irritations, inflammations and healing processes and are inextricably interconnected. Modulators are substances which have a regulating/modulating effect in this complex interaction and thus assist the optimum function of the immune system and/or exert a positive effect on the prophylaxis or treatment of irritations, inflammations and/or wound healing.

The following modulators of physiological processes, such as irritations, inflammation courses and/or wound healing, are known from the state of the art and included by the above-used term modulator:
coenzyme Q10 (Q10, ubiquinone-10). Q10 is essential at a mitochondrial level for optimum function of the immune system (Folkers K, Wolaniuk A: Research on coenzyme Q10 in clinical medicine and in immunomodulation. Drugs Exp. Clin. Res. 1985; 11(8): 539-45). Q10 acts in inflammation processes at the level of gene expression. It exerts inter alia anti-inflammatory effects via influence upon NFkappaB 1-dependent gene expression (Schmeltzer C, Lindner I, Rimbach G, Niklowitz P, Menke T, Döring F, Functions of coenzyme Q10 in inflammation and gene expression, Biofactors 2008; 32(1-4): 179-83). Q10 can be used in a concentration of 1-100 μm, preferably in a concentration of 2-10 μM;
taurine. Taurine is present in immune cells and modulates specific immune cell functions, such as e.g. regulation of inflammatory aspects of the immune response. It also acts as protection in its function as antioxidant (Fazzino F, Obregon F, Lima L. Taurine and proliferation of lymphocytes in physically restrained rats. J. Biomed. Sci. 2010 Aug. 24; 17 Suppl. 1: p. 24) and as osmoregulator (Shioda R., Reinach P. S., Hisatsune T., Miyamoto Y. Osmosensitive taurine transporter expression and activity in human corneal epithelial cells. IOVS, September 2002, Vol. 43, No. 9). Taurine can be used in a concentration of 0.1-50 mM, preferably 0.1-5 mM.
carboxymethyl cellulose (CMC). CMC binds to human epithelial cells and is a modulator of corneal epithelial wound healing (Invest. Ophthalmol. Vis. Sci. 2007 April; 48(4): 1559-67). CMC bonding to the matrix stimulates the adhesion, migration and reepithelialisation of corneal wounds in HCEC;
resolvin (in particular the E and D series). Resolvin E1 (RvE1) induces an increase in cell migration and hence accelerated epithelial wound healing (Zhang et al., IOVS, Vol. 51, No. 11, November 2010); and
protectins. Protectins, like resolvins, are derivatives of eicosapentaenoic acid and docosahexaenoic acid, and exert anti-inflammatory effects (Curr. Opin. Clin. Nutr. Metab. Care. 2011 March; 14(2): 132-7. Docosahexaenoic acid, protectins and dry eye, Cortina M S, Bazan H E).

Surprisingly, it was found that, with the vehicle system according to the invention, in the prophylaxis and/or treatment of diseases of the front and/or back eye portion, in addition, the appearance of tissue irritations and—damage from allergies or inflammations can be efficiently obviated and avoided. Furthermore, in the case of damage which has already occurred, interventions can be made into the inflammation process, an improvement in the healing of possibly present injuries or wounds to the eye epithelium being able to be achieved.

If tissue irritations or—damage are effected, whether by environmental toxins, mechanical irritations, such as friction, pressure, due to bacteria, trauma, chemicals, heat and or excessive immune reactions, such as allergies etc., the result is firstly changes in activity in specific cellular signal paths which in turn lead to specific changes in the gene expression pattern.

In the affected tissues, various inflammation mediators are released and introduce and maintain inflammation processes. The totality of these complex tissue changes is termed inflammation. Inflammations occurring in a regulated manner play an important role in the processes of wound healing.

The preferred, modulator-containing vehicle system, for avoidance, modulation or inhibition, intervenes in these complex processes at various levels:

By means of the vehicle system according to the invention in combination with a modulator, e.g. an inhibitor, antagonist etc., of the NF-KB transcription factor, the production of anti-inflammatory mediators is assisted in order to obviate inflammations prophylactically or therapeutically so that a further improvement in the healing of existing injuries to the eye is assisted since inflammations which affect healing negatively can be suppressed. However, also healing was likewise observed in the case of already existing inflammations.

One of the most important intracellular regulators of inflammation reactions is the transcription factor NF-KB which is activated by various forms of cell stress, for instance chemical-physical toxins, bacterial and viral antigens, cytokines etc., and can change the gene expression in affected cells rapidly and comprehensively. Amongst the genes which are correspondingly highly regulated, there are found in particular cytokines, such as IL-1, TNF-alpha, enzymes, such as COX-2, iNOS, cell adhesion molecules etc., which ensure propagation of the inflammation reaction to other cells and their amplification, often in the sense of a positive feedback.

Modulation of the activation of NF-KB represents a further possibility with which the vehicle system according to the invention can intervene in inflammation events.

Modulators of the NF-kappa B transcription factor can have a direct effect for example on NF-kappa B or indirectly via the signal cascade on NF-kappa B. Antioxidants for example can inhibit components of the NF-kappa B signal transduction path, including the TNF receptor and the proteasome.

By means of the modulation of NF-kappa B, a modulation (in particular suppression) of the tumour-necrosis-factor alpha (TNF-α) can be produced. TNF-α is a signal substance of the immune system and, in the case of diseases, such as e.g. in Sjögren syndrome, keratoconjunctivitis sicca, diseases of the meibomian gland, plays a large part.

During the inflammation process, large quantities of reactive oxygen- and nitrogen species are formed, which intervene inter alia also in a regulatory manner in the inflammation process. Thus, for instance the superoxide radical in immune cells, such as monocytes, macrophages and polymorphonuclear leukocytes, is formed by membrane-resistant NADPH oxidases and is released into the extracellular milieu. In cells activated by inflammatory stimuli, the normally only low superoxide formation increases by a multiple of ten (“oxidative burst”). The formation of this species is responsible not only for killing bacteria but serves also for recruiting leukocytes to the focus of the inflammation and hence has a function for inflammation amplification. Furthermore, reactive oxygen species, at the level of transcription, intervene in the formation of enzymes, e.g. NOS-II. They also activate transcription factors, such as e.g. the NF-KB-B family and protein kinases, whilst they inactivate protein-tyrosine phosphatases.

Also the reactive nitrogen compound nitrogen monoxide is formed, enzymatically and strictly controlled, in a series of tissues. The starting substance is the amino acid L-arginine from which the free radical is formed by the enzyme NO synthestase (NOS). In immune cells, especially in macrophages and granulocytes, the inducible NOS (iNOS) can be expressed after stimulation. Stimuli are thereby above all initiators of inflammation reactions, such as bacteria or components thereof, inflammatory cytokines etc.

Quenching and trapping such reactive oxygen- and nitrogen species can interrupt the reaction chain and thus have a regulatory effect at the transcription and enzyme activation level. In addition, tissue damage, such as e.g. lipidperoxidation by reactive oxygen species can be avoided.

A prophylactic or therapeutic effect is hence effected at various levels of the process.

Thus by formation of a type of protective layer, for example mechanical irritations or contact with allergens or bacteria are avoided. The vehicle system according to the invention therefore preferably comprises correspondingly, for example lipophilic and gel-forming components, consistency providers, viscosity increasing agents which can form a moistening, protective film on epithelia, such as for instance the cornea. Examples of gel formers in oleogels are inter alia the ethylene/propylene/styrene copolymer, butylene/ethylene/styrene copolymer, highly dispersed SiO₂, Al-stearate, Zn-stearate, agar agar (and) alginic acid.

Examples of consistency providers are inter alia, castor oil, jojoba oil, cetylpalmitate, shea butter/cocoa butter, oleyl oleate.

Furthermore, components of the described modulator-containing vehicle system, at one or several levels, e.g. gene transcription, qualitative and/or quantitative modulation of the release of mediator molecules, modulation of the signal transduction etc., intervene in a regulatory manner in for example the process of the inflammation event and/or amplification.

By means of the selection and composition of the ω-3 fatty acids in the vehicle system, the production of anti-inflammatory mediators can be assisted in order to obviate disorders and diseases prophylactically or therapeutically. Examples of diseases are irritations, irritation and swelling of the mucous membranes, eye inflammations, wound healing, treatment of keratoconjunctivitis sicca, of Sjörgen syndrome.

In a preferred vehicle system, the at least one modulator is an inhibitor or antagonist of the NF-KB transcription factor and preferably selected
from natural sources, in particular from the group consisting of allicin, curcumin, EGCD, genistein, melatonin, quercetin, resveratrol, silymarin, sulphoraphanes or mixtures hereof, and/or
from the group consisting of synthetic inhibitors, in particular pyrrolidine dicarbamate, 2-chloro-4-(trifluoromethyl)pyrimidine-5-N-(3′,5′-bis(trifluoromethyl)phenyl)-carboxamide and/or mixtures hereof.

Further examples of modulators of NF-KB activation (antagonists and/or inhibitors) from natural sources are: alpha-lipoic acid (thioctic acid) and dihydrolipoic acid, 2-amino-1-methyl-6-phenylimidazole (4,5b]pyridine (PhIP), N-acetyldopamine dimers (from P. cicadae), allopurinol, anetholdithiolthiones, apocynin, artemesia p7F (5,6,3′5′-tetramethoxy 7,4′-hydroxyflavone), astaxanthin, autumn olive extracts; olive leaf extracts, aventhramides (from oats), bamboo culm extract, benidipine, bis-eugenol, Bruguiera gymnorrhiza compounds, butylated hydroxyanisole (BHA), cepharanthine, caffeic acid phenethyl ester (3,4-dihydroxycinnamic acid, CAPE), carnosol, carotenoids (e.g. beta-carotene), carvedilol, catechol derivatives, Centaurea L (Asteraceae) extracts, chalcone, chlorogenic acid, 5-chloroacetyl-2-amino-1,3-selenazoles, cholestin, chromane-2-carboxylic acid N-substituted phenylamides, polyphenols for example from cocoa or Crataegus pinnatifida, coffee extract (3-methyl-1,2-cyclopentanedione), curcumin (diferulolylmethane); dimethoxycurcumin; ER24 analogue, dehydroepiandrosterone (DHEA) and DHEA sulphate (DHEAS), dibenzylbutyroalactone lignans, diethyldithiocarbamate (DDC), diferoxamine, dihydroisoeugenol; isoeugenol; epoxypseudoisoeugenol-2-methylbutyrate, dihydrolipoic acid, dilazep+feno-fibric acid, dimethyldithiocarbamates (DMDTC), disulfiram, edaravone, EPC-K1 (phosphodiester compound of vitamin E and vitamin C) epigallocatechin-3-gallate (EGCG; green tea polyphenols), ergothioneine, ethylene glycol tetraacetic acid (EGTA), eupatilin, fisetin, flavonoids (Crataegus; Boerhaavia diffusa root; xanthohumol; Eupatorium arnottianum; genistein; camphor oil; quercetin, daidzein; flavones; isorhamnetin; naringenin; pelargonidin; finestin; Sophora flavescens; seabuckthorn fruit berry), sesquiterpene lactones, such as e.g. helenalin, e.g. from arnica extracts, folic acid, gamma-glutamylcysteine synthetase (gamma-GCS), Ganoderma lucidum polysaccharides, garcinol (from extract of Garcinia indica fruit rind), Ginkgo biloba extract, glutathione, guaiacol (2-methoxyphenol), hematein, hinokitiol, hydroquinone, 23-hydroxyursolic acid, IRFI 042 (vitamin E-like compound), iron tetrakis, isoflavones, isosteviol, isovitexin, isoliquiritigenin, kallistatin, kangen-karyu extract, L-cysteine, lacidipines, lazaroids, ligonberries, lupeot, lutein, magnolol, maltol, melatonin, extract of the stem bark of Mangifera indica L., 21(alpha, beta)-methylmelianodiol, 21(alpha,beta)-methylmelianodiol, mulberry anthocyanins, N-acetyl-L-cysteine (NAC), nacyselyn (NAL), nordihydroguaiaritic acid (NDGA), ochnaflavones, onion extract (2,3-dihydro-3,5-dihydroxy-6-methyl-4H-pyranone), orthophenanthrolines, N-(3-oxo-dodecanoyl)homoserine lactones, N-(3-oxo-dodecanoyl)homoserine lactones, paricalcitol, parthenolide, a sesquiterpene lactone, terpenes and a sesquiterpene lactone, parthenolide, phenolic antioxidants (hydroquinone and tert-butylhydroquinone), alkenylphenols from Piper obliquum, alpha-phenyl-n-tert-butyl-nitrone (PBN), phenylarsine oxides (PAO, tyrosine phosphatase inhibitor), Phyllanthus urinaria, phytosteryl ferulates (rice bran), Piper longum linn. extract, Pitavastatin prodelphinidin B2 3,3′di-O-gallate, pterostilbene, pyrrolinedithiocarbamate (PDTC), quercetin, ref-1 (redox factor 1), ref-1 (redox factor 1) rotenone, roxithromycin, rutin, S-allyl-cysteine (SAC, garlic compound), salogaviolide (Centaurea ainetensis), sauchinone, silybin, spironolactone, taxifolin, tempol, tepoxaline (5-(4-chlorophenyl)-N-hydroxy(4-methoxyphenyl)-N-methyl-1H-pyrazole-3-propaneamide), thymoquinone, tocotrienol (palm oil), vanillin (2-hydroxy-3-methoxybenzaldehyde), vitamin B6, a-torphryl succinate, a-torphryl succinate, 2-torphryl acetate, PMC (2,2,5,7,8-pentamethyl-6-hydroxychromane), yakuchinone A and B, zerumbon from types of zingiber (ginger).

As further examples of synthetic sources, the following should be mentioned: cortisones and glucocorticoids, and also the esters thereof, e.g. 16α,17-[(R)-cyclohexylmethylenedioxy]-11β,21-dihydroxypregna-1,4-diene-3,20-dione-21-isobutyrate), salicylanilide inhibitors, 3,4-dihydro-1,1-dimethyl-2H-1,2-benzoselenazine; declopramides and dexlipotam, N-(acetylphenyl)-2-hydroxy-5-iodophenylcarboxamide; N-(2,4-difluorophenyl)-2-hydroxy-5-nitrophenylcarboxamide; N-(2,4-difluorophenyl)-2 hydroxy-5-iodophenylcarboxamide, or a pharmaceutically acceptable salt, hydrate or solvate thereof.

Also in addition modulators of COX-2 can be contained, such as e.g. basil, berberine, curcumin, EGCG, ginger, hops (Humulus lupulus), fish oil, oregano, quercetin, resveratrol, rosemary.

Particularly preferred active substances which are used in the ophthalmological vehicle system according to the invention are anti-inflammatory substances, such as acetylsalicylic acid and derivatives such as L-lysin, antiseptic substances, such as bibrocathol, antibiotics such as ampicillin, sulfacetamide, doxycyclin, gentamycin or ciprofloxacin, anti-allergic agents such as cromoglicic acid, antihistamines such as levocabastine, azelastine and/or dexpanthenol.

The ophthalmological vehicle system according to the invention is preferably preservative-free, e.g. free of thimerosal etc. In the case where preservatives are contained, it is particularly preferred and sufficient that merely silver ion vehicle systems are present. The silver ions can thereby be contained in a preferred concentration range of 1 ppb to 2 ppm, further preferred from 10 ppb to 1 ppm. The presence of silver ions can result due to the external addition of silver salts, such as e.g. silver nitrate, to the vehicle system, but can also result from the fact that the vehicle system is in contact with an object made of a silver alloy or solid silver, as a result of which a small proportion of the silver is dissolved and is transferred into the vehicle system. Possible silver-containing objects can be for example silver spirals, as occur in metering devices for liquids or pastes. Corresponding metering devices are known, for example from the patent applications EP 1 466 668 A1. The above-mentioned low silver concentrations are sufficient to exert a preserving effect, e.g. a bactericidal effect on the vehicle system.

In particular in the case where the vehicle system comprises silver ions or the intention is to bring the vehicle system in contact with a silver-containing object, it is advantageous to add to the vehicle system at least one complex former, selected from the group of sulphur-containing organic compounds, in particular sodium thiosalicylic acid, thiosorbit, cysteine, N-acetyl-L-cysteine, cysteinehydrochloride, cysteamine, cystine, methionine, glutathione, S-acetylglutathione, thioglycerol, thiourea, thiolactate; and/or EDTA, EGTA and also combinations hereof, preferably in a quantity of 0.0001-5% by weight, preferably 0.0001-1% by weight, preferably 0.001-0.5% by weight, relative to the total vehicle system.

As an alternative hereto, the vehicle system can however also comprise preservatives, for example at least one preservative which is common in ophthalmology, in a quantity of 0.001-1% by weight, preferably 0.01-0.5% by weight, preferably 0.01-0.04% by weight, relative to the total composition, preferably a preservative selected from the group consisting of polyquad, sodium perborate, purite; alcohols, such as chlorobutanol, benzyl alcohol, phenoxyethanol; carboxylic acids, such as sorbic acid; phenols, such as methyl-/ethylparaben; amidines, such as chlorohexidinedigluconate; quaternary ammonium compounds, such as benzalkonium chloride, benzethonium chloride and cetylpyridinium chloride, benzyl bromide and/or combinations hereof.

The vehicle system according to the present invention can comprise in addition one or more of the following components:

a) at least one anti-inflammatory- and/or anti-oxidatively- and/or anti-allergically-acting substance, selected from the group consisting of flavonoids (e.g. rutin, quercetin, curcurmin), isoflavonoids (e.g. silymarin), polyphenols (e.g. resveratol), anthocyanes, triterpenes, monoterpene alcohols, phenolcarboxylic acids, carotenoids (e.g. β-carotene, α-carotene, lycopin, β-cryptoxanthine, lutein, zeaxanthin), retinoids (e.g. tretinoin), tocopherols (vitamin E) and biotin, vitamins A, C, D, K, coenzyme Q (=Q10) carnitine, N-Acetyl-carnitine, glutathione, carnesol, ubiquinone and/or taurine and/or plant single substances, substance mixtures, a liquid or solid extract, a distillate or an oil or etheric oil, preferably from plants of the genus or species of rosemary, sea buckthorn, myrrh, eupharis (eyebright), camomile, arnica, marigold, thyme, echinacea, calendula, tea tree, tea bush, chokeberry (aronia), ginkgo, ginseng, blueberries, elderberries, lavender, anise, preferably in a quantity of 0.01 and 5% by weight, relative to the total composition,

b) at least one gel former, selected from the group consisting of natural or synthetic polymers, preferably in a quantity between 0.01 and 5% by weight, relative to the total composition,

c) at least one thickener, preferably in a quantity between 0.5 and 5% by weight, relative to the total composition,

d) at least one moisture-retaining means,

e) at least one auxiliary agent, selected from the group consisting of inorganic buffer substances, organic buffer substances, inorganic salts, organic salts, viscosity regulators, solvents, solubility promoters (e.g. lecithin, macrogolglycerol monostearates, macrogolglycerol ricenoleate, polyethylene monostearate (e.g. Myrj 49), polysorbates, glyceryl monooleates, glyceryl monostearates, glyceryllaurate, methyl cellulose, polychol, sorbitan monolaurate, sorbitan oleate, sorbitan palmitate, sorbitan trioleate, inter alia,

f) solution accelerators, salt formers, viscosity- and consistency controllers, solubilisers, wetting agents, extenders, filling and carrier substances, osmolarity regulators and also mixtures thereof, and also

g) combinations of the previously mentioned components.

The at least one anti-inflammatory- and/or anti-oxidatively- and/or anti-allergically-acting substance is used primarily because of its physiological effect in the eye. These substances stop for example oxidative damage in the tissue, e.g. caused by reactive oxygen species in the case of excessive immune reactions; they act in part to be anti-inflammatory etc. In addition, they also protect Omega-3 FS against oxidation in the formulation before oxidative decomposition.

Vitamin A hereby comprises the vitamin A1 (retinol), vitamin A2 (3-dehydroretinol), vitamin A acid, vitamin-A derivatives (retinylpalmitate, retinylacetate etc.,) all-trans-retinolic acid (ATRA, aRA, tretinoine), 13-cis-retinolic acid or -retinoic acid (isotretinoine), vitamin A analogues, such as e.g. the all-trans retinoic acid.

Vitamin C comprises ascorbic acid, ascorbylpalmitate and ascorbylacetate and vitamin E comprises gamma-tocotrienol and 6-hydroxy-2,5,7,8-tetramethylchroman-2-carboxylic acid (trolox).

Examples of antioxidants are terpenoids (monoterpenoid), sesquiterpenoid, diterpenoid, triterpenoid), carotenoids (α- and β-carotene), hydroxytyrosol, zeathin, lutein, lycopenes, anthocyanins, cryptoxanthins, xanthophylls, epicatechin, quercetin, punicalagins and ellagic acid; chlorogenic acid, gallic acid, ferulic acid, caffeic acid, a-tocopherol, a-tocopherol ester, ascorbic acid, ascorbic acid ester (myristate, -palmitate and -stearate), β-carotene, cysteine, acetylcysteine (N-acetyl-L-cysteine also represents a mucolytic means at the same time), coenzyme Q, idebenones (synthetic quinones similar to Q10), folic acid (vitamin B2 group) phytic acid, cis- and/or trans-urocanic acid, carnosine (N-β-alanine-L-histidine), histidine, flavones, flavonoids, lycopin, taurine, tyrosine, gluthation, gluthation ester, α-lipoic acid, ubiquinone, niacin, nordihydroguaiaretic acid, gallic acid ester (ethyl-, propyl-, octyl-, dodecylgallate), phosphoric acid derivatives (monophosphates, polyphosphates), butylhydroxytoluene, butylhydroxyanisole, tetraoxydimethylbiphenyl, tocotrienoles (part of the vitamin E substance group), polyalcohols, polyphenols, citric acid, tartaric acid, edetic acid (EDTA as DiNa- or DiNaCa salt), coniferylbenzoate and/or derivatives thereof as antioxidants.

The antioxidants can be added directly or in the form of oils or etheric oils.

Wheat germ oil comprises for example tocopherols, carotenoids, ergocalciferol, folic acid (vitamin B9), pantothenic acid, phytosterols and phenols, such as dihydroquercetin etc.

Gel formers suitable for the vehicle system according to the invention preferably comprise natural or synthetic polymers. Natural polymers are preferably selected from the group consisting of agar-agar, alginic acid, alginate, amidated pectin, propylene glycol alginate, carbomer, carrageenan, casein, cellulose derivatives (methyl-, hydroxyethyl, carboxymethyl cellulose sodium), dammar gum, dextrins, furcellaran, gelatines, guar gum, guar gum, gellan gum, ghatti gum, Gummi arabicum, gum from spruce sap, carob bean gum, karaya gum, keratin, konjak flour, L-HPC, locust bean gum, mastic, pectin, shellac, (possibly modified) starch, tara gum, traganth, xanthan gum and derivatives thereof.

Thickeners which can be contained preferably in the vehicle system according to the invention comprise for example candelilla and carnauba wax and also microcrystalline waxes, carbomer, polyethylene oxide thickeners, polaxamers, hydroxylethyl cellulose, hydroxypropyl cellulose, hypromellose, povidone, hyaluronic acid, polylactic acid and derivatives thereof. The thickener is preferably used in a quantity of 0.5 to 5% by weight, relative to the total weight of the pharmaceutical composition according to the invention.

Preferred formulations in which the ophthalmological vehicle system of the present invention can be present are thereby in liquid, viscous or semi-solid form, in particular in the form of a gel, a thixotropic gel, a lipogel, an oleogel, an organogel, a microemulsion gel, a spray gel, a water-in-oil emulsion, in particular a water-in-oil micro- or -nanoemulsion, an in situ gel, a cream or an oil.

Microemulsion gels are thereby emulsion gels in which the average particle diameter d₅₀ is less than 0.1 μm.

The ophthalmological vehicle system of the present invention is suitable in particular for the prophylaxis and/or treatment of inflammation (e.g. uveitis, uveitis anterior, uveitis intermedia, uveitis posterior, panuveitis), iritis, chorioiditis, azoor (acute zonal occult outer retinopathy), neuritis nervi optici) cataract (grey star), glaucoma, retinopathy, macular degeneration (AMD), retinal detachment, retinoblastoma and/or choroid melanoma and/or for pre- and/or post-treatment of surgical operations on the eye, in particular surgical operations selected from the group consisting of surgical operations on the front eye portion, cataract extraction with lens implants, refractive-surgical operations, operations on the cornea and corneal transplants and/or operations on the sclera.

Possibilities for application of the ophthalmological vehicle system are thereby in particular topical applications to the eye, e.g. by putting drops in the eye or on the eye surface, spraying in or on the eye or onto the eye surface, or by putting in drops, as gel deposit into the conjunctival sac or as insert.

Preferably, the vehicle system of the present invention can be applied once daily to once hourly, preferably once to four times daily.

In addition, the present invention relates to an ophthalmological kit, comprising
a) an ophthalmological system comprising ≧30 to 99.5% by weight, relative to the total ophthalmological kit, of at least one fatty acid ester and 0.001% by weight to ≦50% by weight, relative to the total ophthalmological kit, of one or at least one emulsifier and also
b) an ophthalmological active substance formulation, comprising at least one ophthalmological active substance, selected from the group consisting of antibiotics, corticoids, local anaesthetics, decongestants, non-steroidal antiphlogistics, virustatics, antiseptics, cortisone, anti-allergic active substances, prostaglandin analogues, active substances from the active substance class of antihistamines and/or of corticosteroids, anti-allergic active substances, pantothenic acid derivatives, non-steroidal anti-inflammatory drugs, vascoconstrictors and/or anti-glaucoma active substances in a pharmaceutically effective concentration,
   as separate formulations.

The ophthalmological kit according to the present invention is thereby suitable for the same indications as the previously described ophthalmological vehicle system. In contrast to the ophthalmological vehicle system, the kit consists of at least two separate components, i.e. formulations, which can be applied to the eye for example simultaneously with each other, but also in succession in principle in any sequence, however preferably the vehicle system first. The application possibilities are thereby likewise identical to the previously described ophthalmological vehicle system.

In a preferred embodiment, the ophthalmological system, as component of the ophthalmological kit, is free of active substances, i.e. the active substance is provided separately in the kit and in addition to the ophthalmological system. This enables a broad application band width so that a particularly targeted application of the individual active substances to the eye together with the ophthalmological system can be achieved for improving the permeation of the active substance. In addition, optimal storage conditions for the individual formulations can be chosen separately. Thus, the ophthalmological vehicle system can be stored in preferred storage conditions, in addition likewise the ophthalmological active substance formulations in optimal storage conditions for this so that in total long durability of the total ophthalmological kit can be ensured.

Likewise, it is conceivable that the ophthalmological system comprises in fact one or more ophthalmological active substances as a component of the kit, also at least one further active substance being able to be combined in addition with the additional ophthalmological active substance formulation.

The components of the kit can be mixed together for example immediately before application to the eye and the mixture can be applied on or in the eye by the preferred types of application described further back; likewise, also individual application of the components of the kit on or in the eye is however possible. In particular, it is preferred to apply the ophthalmological system first so that removing the tight junctions of the epithelia at the respective application site is effected. Subsequently, the active substance composition can be applied.

The ophthalmological system of the kit can thereby be configured identically to the previously described ophthalmological vehicle system, in particular as concerns its consistency or possibly present further ingredients. In this respect, reference is made to the preferred embodiments of the ophthalmological vehicle system which are described further back and apply analogously to the ophthalmological system of the kit.

All active substances mentioned previously in the context of the ophthalmological vehicle system are suitable similarly as suitable active substances for the ophthalmological kit.

The ophthalmological active substance formulation in the kit system can comprise “sustained-release” drug-delivery systems, such as e.g. degradable polymers, microspheres, micelles, liposomes.

The ophthalmological active substance formulation in the kit system can be present in aqueous or oily formulation, as hydrogels, oleogels, spray gels, microemulsions and the like.

The kit can also be combined from an ophthalmological system for increasing permeation and a commercial active substance preparation.

The subject of the present invention is likewise the use of a composition, comprising
a) ≧30 to 99.95% by weight, relative to the total composition, of at least one fatty acid ester,
b) 0.05% by weight to ≦50% by weight, relative to the total composition, of one or at least one emulsifier,
c) at least one ophthalmological active substance, selected from the group consisting of antibiotics, corticoids, local anaesthetics, decongestants, non-steroidal antiphlogistics, virustatics, antiseptics, cortisone, anti-allergic active substances, prostaglandin analogues, active substances from the active substance class of antihistamines and/or of corticosteroids, anti-allergic active substances, pantothenic acid derivatives, non-steroidal anti-inflammatory drugs, vascoconstrictors, and/or anti-glaucoma active substances in a pharmaceutically effective concentration,
   and/or a previously described ophthalmological kit as vehicle system, penetration accelerator, penetration enhancer, absorption enhancer/-improver/-accelerator for the permeation and/or for the active substance transport of ophthalmological active substances through the cornea and/or the sclera of the eye of mammals.

The previously described composition is thereby applied in the same way and/or for the same application possibilities, as described previously for the ophthalmological vehicle system or the ophthalmological kit. The preferred substance compositions of the above-described composition used thereby correspond likewise to those of the ophthalmological vehicle system, as described above.

The invention relates in addition to a fluid dispenser for a sterile fluid, having
a) a passage which connects an inlet opening for a fluid contained in a storage container made of a flexible material and an outflow opening for dispensing the fluid and has therein at least one oligodynamically active substance which is in contact with the fluid;
b) a metering pump which operates without compressed air compensation, comprising an inlet valve for closing the inlet opening, the inlet valve having a material which can interact with the fluid via an oligodynamically active substance; and
c) a spring mechanism which can be in contact with the fluid,
   the inlet valve and the spring mechanism having a stainless steel material as an oligodynamically active substance and a decontamination mechanism being provided in the upper part of the outlet channel, the decontamination mechanism having a material which can interact with the fluid via an oligodynamic substance which is selected from the group consisting of silver, silver salts, other silver compounds, alloys and nanomers thereof in either metallic or salt form or as a chemical compound thereof, the fluid contained in the storage container being an ophthalmological vehicle system according to the invention.

In the case of the fluid dispenser according to the invention, the spring can be in contact with the fluid, for example by the spring being located in the passage.

With respect to such a fluid dispenser in which the ophthalmological vehicle system can be stored according to the invention, reference is made to the patent application EP 1 466 668 A1. All embodiments with respect to the fluid dispenser of this patent application are made by reference also the subject of this patent application.

Likewise, it can however be provided that the spring cannot come in contact or does not come in contact with the fluid. According to this likewise preferred embodiment, the spring is not located for example in the passage, e.g. disposed outside the passage.

The present invention is explained in more detail with reference to the subsequent formulations, given by way of example, without restricting the invention to the special parameters represented there.

The following constituents according to the invention, given by way of example, are suitable as ophthalmological vehicle system for the permeation and/or the active substance transport of ophthalmological active substances through the cornea and/or the sclera of the eye of mammals for the prophylaxis and/or treatment of diseases of the front and/or back eye portion.

### EXAMPLE 1

### Transparent Microemulsion Gel Base for the Formulation of Various Active Substances

| Components | Quantity (% by wt) |
|---|---|
| Epikuron 200 | 7.5% |
| isopropyl myristate | 92% |
| DHA/EPA | 0.1% |
| alternatively also Q10 | 0.05% |
| water | 0.4% or 0.45 until gel formation |

### EXAMPLE 2

### Transparent Microemulsion Gel Base for the Formulation of Various Active Substances

| Components | Quantity (% by wt) |
|---|---|
| Epikuron 200 | 7.5% |
| isopropyl palmitate | 92% |
| DHA/EPA | 0.1% |
| alternatively also Q10 | 0.05% |
| water | 0.4% or 0.45 |
| | until gel formation |

### EXAMPLE 3

### Transparent Microemulsion Gel Base for the Formulation of Various Active Substances

| Components | Quantity (% by wt) |
|---|---|
| Epikuron 200 | 7.5% |
| isopropyl myristate | 87.05% |
| alginic oil | 5% |
| alternatively also Q10 | 0.05% |
| water | 0.4% or 0.45 until |
| | gel formation |

### EXAMPLE 4

### Oily Base for the Formulation of Various Active Substances

| Components | Quantity (% by wt) |
|---|---|
| lecithin, e.g. Epikuron 100 or 200 | 0.3% to 6%, e.g. 3% |
| isopropyl myristate | up to 100% |
| rapeseed oil, alginic oil and/or perilla oil | 0.1% to 2%, e.g. 0.5% |
| alternatively also tocopherol | 0.05% |

### EXAMPLE 5

### Oily Base for the Formulation of Various Active Substances

| Components | Quantity (% by wt) |
|---|---|
| lecithin, e.g. Epikuron 100 or 200 | 0.05% to 1%, e.g. 0.5% |
| isopropyl myristate | up to 100% |
| DHA/EPA | 0.01 to 0.2%, e.g. 0.2% |
| ascorbic acid palmitate | 0.01% to 0.1%, e.g. 0.05% |
| tocopherol | 0.005% to 0.05%, e.g. 0.075% |

### EXAMPLE 6

### Oily Base for the Formulation of Various Active Substances

| Components | Quantity (% by wt) |
|---|---|
| lecithin, e.g. Epikuron 100 or 200 | 1 to 8%, e.g. 5% |
| isopropyl palmitate | up to 100% |
| castor oil | 3 to 20%, e.g. 15% |
| carnesol and/or Q10 | 0.01% to 0.2%, e.g. 0.05% |
| triglycerol diisostearate | 0.3% to 2%, e.g. 1% |
| possibly water | 0.1% to 0.5% |

### EXAMPLE 7

### Oily base for the formulation of various active substances

| Components | Quantity (% by wt) |
|---|---|
| lecithin and/or isotridecylglycidyl | 0.8% to 15%, e.g. 1% |
| succinate | |

| | |
|---|---|
| isopropyl palmitate | up to 100% |
| castor oil | 0.2 to 10% |
| carnesol | 0.001 to 0.1%, e.g. 0.05% |
| triglycerol diisostearate | 0.1% to 5%, e.g. 1% |

The effectiveness of the ophthalmological vehicle system according to the invention is verified by the subsequent tests.

### EXAMPLE 8

### Oily Base for the Formulation of Various Active Substances

| Components | Quantity (% by wt) |
|---|---|
| Epikuron 100 | 0.5-4 |
| tocopherol | 0.005 |
| ascorbyl palmitate | 0.010 |
| isopropyl myristate | up to 100% |

### EXAMPLE 9

### Oily base for the formulation of various active substances

| Components | Quantity (% by wt) |
|---|---|
| Epikuron 100 | 4 |
| isopropyl myristate | 95.99 |
| tocopherol | 0.005 |
| ascorbyl palmitate | 0.010 |
| alternatively zeaxanthin, lutein, | 0.01-0.5 |
| rutin, or other antioxidants | |

### EXAMPLE 10

### Water-in-Oil Emulsion as Base for the Formulation of Various Active Substances

| Components | Quantity (% by wt) |
|---|---|
| PEG-7 hydrogenated castor oil | 0.5-5 |
| ricinus oil | 0.05-2 |
| isopropyl myristate | 60 |
| water | 0.05-2 |
| alternatively lanolin alcohol, | 0.05-2 |
| ricinus oil, Sali Salix extract | |

### EXAMPLE 11

### Oil or Microemulsion as Base for the Formulation of Various Active Substances

| Components | Quantity (% by wt) |
|---|---|
| lecithin and/or isostearyl-diglyceryl succinate | 0.8-15, e.g. 1 |
| (e.g. Imvitor 742 or 780) | |

| | |
|---|---|
| isopropyl myristate | 20 to 40, e.g. 31 |
| miglyol | up to 100% |
| water | 0 to 10 |

### EXAMPLE 12

### Oil or Microemulsion as Base for the Formulation of Various Active Substances

| Components | Quantity (% by wt) |
|---|---|
| ricinus oil | 2.5 to 7 |
| isopropyl myristate | 31 to 50 |
| miglyol (e.g. Miglyol 812) | up to 100% |
| benzyl alcohol | 0.5 to 1 |
| isostearyl diglyceryl succinate | 0.5 to 7 |
| (e.g. Imvitor 742 or 780) | |

### EXAMPLE 13

### Oil or Microemulsion as Base for the Formulation of Various Active Substances

| Components | Quantity (% by wt) |
|---|---|
| isopropyl myristate | 20 to 40, e.g. 30.5 |
| miglyol (e.g. Miglyol 812) | up to 100% |
| benzyl alcohol | 0.5 to 1 |
| possibly lecithin (e.g. Epikuron 100 or 200) | 0.5 to 7.5 |
| possibly water | 0.2 to 0.5 |

### EXAMPLE 14

### Cream as Base for the Formulation of Various Active Substances

| Components | Quantity (% by wt) |
|---|---|
| triglycerine diisostearate | 10 to 35 |
| isopropyl myristate | 30.5 to 35 |
| mixture of paraffinum liquidum or | up to 100% |
| subliquidum and polyethylene (e.g. MR 21,000) | |
| in the weight ratio 80:20 to 99:1, e.g. 95:5 | |

All formulations according to examples 1 to 14, given by way of example, are likewise suitable as ophthalmological system in an ophthalmological kit according to the present invention.

### EXAMPLE 15

### Kit System

1) Ophthalmological system

| Components | Quantity (% by wt) |
|---|---|
| lecithin, e.g. Epikuron 100 and/or 200 | 0.5 to 7.5% |
| isopropyl myristate | up to 100% |
| alternatively, also subsequent substances | |
| can be contained: | |

| | |
|---|---|
| water | 0.1 to 0.5 |
| DHA/EPA, rapeseed oil and/or perilla oil | 0.01 to 0.5% |

2) Active substance formulation

| Components | Quantity (% by wt) |
|---|---|
| variable active substance, e.g. hydrophilic or | prescribed dose |
| lipophilic active substances | |
| macrogolglycerol ricenoleate | 0.1 to 5% |
| water | up to 100% |
| alternatively, also subsequent substances | |
| can be contained: | |
| DHA/EPA, rapeseed oil and/or perilla oil | 0.01 to 0.5% |
| carotenoids, e.g. beta-carotene | 0.01 to 0.2% |

All the above-described active substances are suitable as active substances.

I. EVEIT Test

The subsequent experiments verify the permeation capacity of the ophthalmological vehicle system according to the invention. Fluorescein was hereby tested as substance to be introduced into the eye, by way of example. The substances used are thereby:

Culture Medium:

Ringer's solution: Ringer's solution is an isotonic electrolyte solution which is used inter alia as culture medium for fresh tissue. The standard Ringer's solution comprises, to 1,000 ml aqua destillata, 8.6 g sodium chloride, 0.3 g potassium chloride, 0.33 g calcium chloride.

Hylo-COMOD®:

HYLO-COMOD® is a sterile, preservative-free solution with 1 mg/ml sodium hyaluronate, a citrate buffer, sorbitol and water and is marketed by URSAPHARM Arzneimittel GmbH.

Ursaphama A:

Microemulsion gel on a water-in-oil base, comprising 7.5% by weight of lecithin (Epikuron 200), 0.4% by weight of water, 0.1% by weight of a mixture of eicosapentaenoic acid ethyl ester/docosapentaenoic acid ethyl ester (weight ratio approx. 73:27) and also ad 100% isopropyl myristate.

Ursapharm C:

Microemulsion gel on an oil-in-water basis, comprising 0.1% by weight of a mixture of eicosapentaenoic acid ethyl ester/docosapentaenoic acid ethyl ester (weight ratio approx. 73:27), 1% by weight of macrogolglycerol ricinoleate (e.g. Cremophor EL), 0.1% by weight of hyaluronic acid, <0.3% by weight of mixed tocopherols, 0.05% by weight of Q10, 3.2% by weight of sorbitol, 0.005% by weight of citric acid, 0.85% by weight of sodium citrate and also ad 100% by weight of water.

### EXPERIMENT 1

### Negative Control—Merely Simulation of the Blink by Application of Culture Medium

Preparation and cultivation of a rabbit cornea according to the EVEIT system. The preparation was effected at most 8 h post mortem. Cultivation was effected at 32° C. and air humidity of >95%. A simulation of the blink was effected by application of culture medium (38 μl) on the corneal apex at a time interval of 60 min. 24 h after preparation, the culture was subjected to a quality test (macroscopically including fluorescein dye and also by optical coherence tomography (OCT)) which verifies successful culturing with physiological vitality of the organ culture. Subsequently, the cornea was further cultivated over an observation period of 3 days whilst maintaining application of culture medium every 60 minutes. Documentation of the test course was effected daily by macroscopy and OCT. The corneal cultures were incubated once daily for 60 seconds with 100 μl of an aqueous solution of sodium fluorescein (5 mg/ml). Subsequently, the fluorescein was rinsed thoroughly from the corneal surface with Ringer's solution. After rinsing, the corneal cultures were left to stand for 60 minutes in the incubator at 32° C. and 100% air humidity. In this time window, no exchange of culture medium was effected in the anterior chamber. The anterior chamber volume was exchanged completely after 60 minutes waiting time. The substance quantity of fluorescein found at this time in the artificial anterior chamber was determined photometrically.

Result: In the OCT, no structural change in the epithelium or in the stroma is detectable within the culture time. In the course of the culture, swelling of the cornea can be observed 48 hours after first application. This increase in layer thickness in the range of 30% is a typical observation within the culture which is in the physiological range and indicates merely a somewhat increased water absorption of the stroma under the culture conditions. Macroscopically, likewise no striking feature is present. The green colouration of the macroscopies is caused by small quantities of fluorescein in the artificial anterior chamber which result from the permeation measurements implemented in parallel. The epithelia themselves are fluorescein-negative.

### EXPERIMENT 2

### Drop Application Every 60 Minutes, HYLO-COMOD®

Preparation and cultivation of a rabbit cornea according to the EVEIT system. The preparation was effected at most 8 h post mortem. Cultivation was effected at 32° C. and an air humidity of >95%. A simulation of the blink was effected by application of culture medium (38 μl) on the corneal apex at a time interval of 60 min. 24 h after preparation, the culture was subjected to a quality test (macroscopically including fluorescein dye and also by optical coherence tomography (OCT)) which verifies successful culturing with physiological vitality of the organ culture. Subsequently, the test substance HYLO-COMOD® was applied (38 μl) on the cornea, repeated at a time interval of 60 min, over a period of 3 days. Application of the test substance was thereby effected alternately, after 30 minutes, with application of the culture medium. Documentation of the test course was effected daily by macroscopy and OCT. The corneal cultures were incubated once daily for 60 seconds with 100 μl of an aqueous solution of sodium fluorescein (5 mg/ml). Subsequently, the fluorescein was rinsed thoroughly from the corneal surface with Ringer's solution. After rinsing, the corneal cultures were left to stand for 60 minutes in the incubator at 32° C. and 100% air humidity. In this time window, no exchange of culture medium was effected in the anterior chamber. The anterior chamber volume was exchanged completely after 60 minutes waiting time. The substance quantity of fluorescein found at this time in the artificial anterior chamber was determined photometrically.

Result: In the OCT, no structural change in the epithelium as a result of the application is detectable within the culture time. In the course of the culture, swelling of the cornea can be observed 48 hours after first application. This increase in layer thickness is a typical observation within the culture which is in the physiological range and indicates merely a somewhat increased water absorption of the stroma under the culture conditions. Macroscopically, likewise no striking feature is present. The green colouration of the macroscopies is caused by small quantities of fluorescein in the artificial anterior chamber which result from the permeation measurements implemented in parallel. All epithelia are fluorescein-negative.

### EXPERIMENT 3

### Positive Control—Drop Application Every 60 Min, Benzalkonium Chloride 0.001% in Ringer's Solution

Preparation and cultivation of a rabbit cornea according to the EVEIT system. The preparation was effected at most 8 h post mortem. Cultivation was effected at 32° C. and an air humidity of >95%. A simulation of the blink was effected by application of culture medium (38 μL) on the corneal apex at a time interval of 60 min. 24 h after preparation, the culture was subjected to a quality test (macroscopically including fluorescein dye and also by optical coherence tomography (OCT)) which verifies successful culturing with physiological vitality of the organ culture. Subsequently, the test substance BAC 0.001% was applied (38 μl) on the cornea, repeated at a time interval of 60 min, over a period of 3 days. Application of the test substance was thereby effected alternately, every 30 minutes, with application of the culture medium. Documentation of the test course was effected daily by macroscopy and OCT. The corneal cultures were incubated once daily for 60 seconds with 100 μl of an aqueous solution of sodium fluorescein (5 mg/ml). Subsequently, the fluorescein was rinsed thoroughly from the corneal surface with Ringer's solution. After rinsing, the corneal cultures were left to stand for 60 minutes in the incubator at 32° C. and 100% air humidity. In this time window, no exchange of culture medium was effected in the anterior chamber. The anterior chamber volume was exchanged completely after 60 minutes waiting time. The substance quantity of fluorescein found in the artificial anterior chamber was determined photometrically.

Result: In the OCT, a significant reduction in the epithelium layer thickness is detectable 48 hours after the first application of BAC 0.001%. 72 h after the first application, the epithelium has almost disintegrated structurally and no longer forms a closed layer. Because of this epithelial defect, the swelling of the cornea at this time is significantly above the dimension observed otherwise under physiological conditions. The green colouration of the macroscopies is caused in part by small quantities of fluorescein in the artificial anterior chamber which result from the permeation measurements implemented in parallel. 48 hours and 72 hours after the first application, the epithelium is however significantly fluorescein-positive.

### EXPERIMENT 4

### Drop Application Every 60 Min, Ursapharm a (Lecithin Gel+IPM 0.4%)

Preparation and cultivation of a rabbit cornea according to the EVEIT system. The preparation was effected at most 8 h post mortem. Cultivation was effected at 32° C. and an air humidity of >95%. A simulation of the blink was effected by application of culture medium (38 μl) on the corneal apex at a time interval of 60 min. 24 h after preparation, the culture was subjected to a quality test (macroscopically including fluorescein dye and also by optical coherence tomography (OCT)) which verifies successful culturing with physiological vitality of the organ culture. Subsequently, the test substance Ursapharm A was applied (38 μl) to the cornea, repeated at a time interval of 60 min, over a period of 3 days. Application of the test substance was thereby effected alternately, every 30 minutes, with application of the culture medium. Documentation of the test course was effected daily by macroscopy and OCT. The corneal cultures were incubated once daily for 60 seconds with 100 μl of an aqueous solution of sodium fluorescein (5 mg/ml). Subsequently, the fluorescein was rinsed thoroughly from the conical surface with Ringer's solution. After rinsing, the conical cultures were left to stand for 60 minutes in the incubator at 32° C. and 100% air humidity. In this time window, no exchange of culture medium was effected in the anterior chamber. The anterior chamber volume was exchanged completely after 60 minutes waiting time. The substance quantity of fluorescein found in the artificial anterior chamber at this time was determined photometrically.

Result: In the test, the cornea with an initial thickness of 405 swells significantly to 649 μm over the test period. The epithelial complex is no longer detectable at the end. Correspondingly in the fluorescein dye, complete central colouration of the cornea is found. The picture corresponds to the positive control.

### EXPERIMENT 5

### Drop Application Every 60 Min, Ursapharm C

Preparation and cultivation of a rabbit cornea according to the EVEIT system. The preparation was effected at most 8 h post mortem. Cultivation was effected at 32° C. and an air humidity of >95%. A simulation of the blink was effected by application of culture medium (38 μl) on the corneal apex at a time interval of 60 min. 24 h after preparation, the culture was subjected to a quality test (macroscopically including fluorescein dye and also by optical coherence tomography (OCT)) which verifies successful culturing with physiological vitality of the organ culture. Subsequently, the test substance Ursapharm C was applied (38 μl) on the cornea, repeated at a time interval of 60 min, over a period of 3 days. Application of the test substance was thereby effected alternately, every 30 minutes, with application of the culture medium. Documentation of the test course was effected daily by macroscopy and OCT. The corneal cultures were incubated once daily for 60 seconds with 100 μl of an aqueous solution of sodium fluorescein (5 mg/ml). Subsequently, the fluorescein was rinsed thoroughly from the corneal surface with Ringer's solution. After rinsing, the corneal cultures were left to stand for 60 minutes in the incubator at 32° C. and 100% air humidity. In this time window, no exchange of culture medium was effected in the anterior chamber. The anterior chamber volume was exchanged completely after 60 minutes waiting time. The substance quantity of fluorescein found in the artificial anterior chamber at this time was determined photometrically.

Result: In the OCT, no structural change in the epithelium as a result of the application is detectable within the culture time. In the course of the culture, swelling of the cornea can be observed 48 hours after the first application. This increase in layer thickness is a typical observation within the culture which is in the physiological range and only indicates a somewhat increased water absorption of the stroma under the culture conditions. Macroscopically, likewise no striking feature is present. The green colouration of the macroscopies is caused by small quantities of fluorescein in the artificial anterior chamber which result from the permeation measurements implemented in parallel. All epithelia are fluorescein-negative.

II. Quantitative Evaluation of the Fluorescein Permeation into the Artificial Anterior Chamber with Drop Application

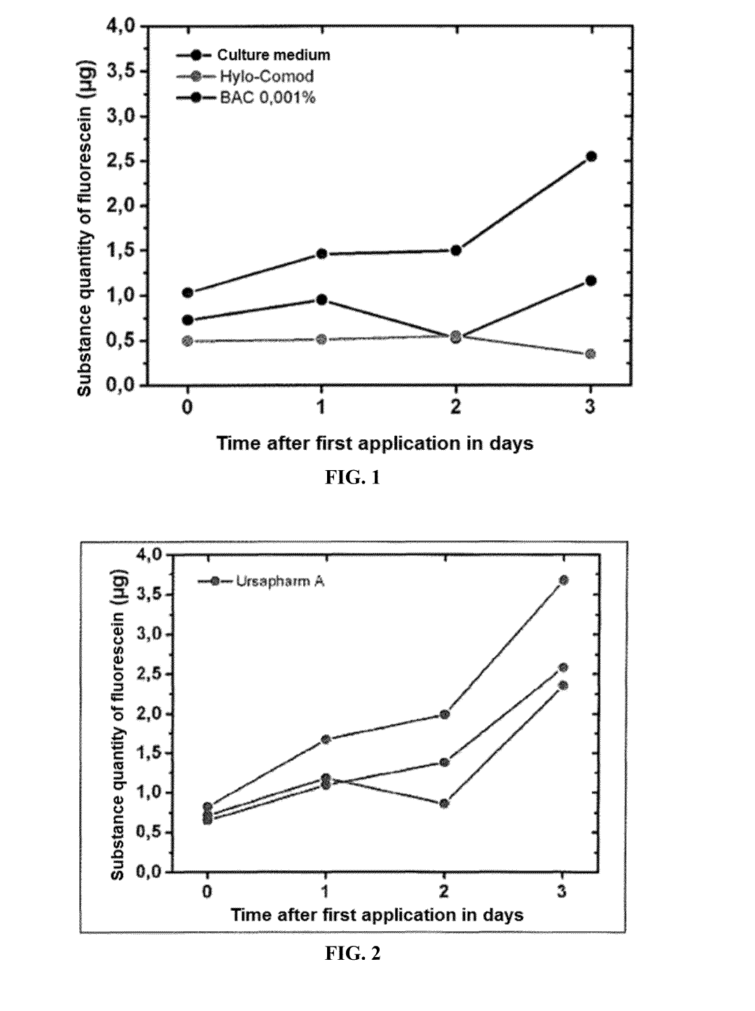

Fluorescein permeation: control substances (culture medium (Experiment 1) HYLO-COMOD® (Experiment 2) and benzalkonium chloride 0.001% (Experiment 3); FIG. 1)

In FIG. 1, the established substance quantity of fluorescein within the simulated eye anterior chamber is plotted over time after the first application of the listed control substances. The initial value at the time zero was measured directly before the first application. Subsequently, a further value was determined every 24 hours. If only culture medium is applied in drops onto the corneal epithelium at a time interval of 60 minutes for simulation of the blink, then the permeability of the cornea increases over the culture duration of 3 days by approx. 50%. If between the simulation of the blink in addition HYLO-COMOD® is applied in drops onto the cornea, then this increase can be completely suppressed. If instead of HYLO-COMOD® the permeation enhancer benzalkonium chloride (0.001%) is applied on the cornea alternately with the application with culture medium, then the measured permeation of fluorescein increases significantly by more than 150 percent. The test indicates that there is a low permeation of fluorescein through the epithelium at all times of the EVEIT, as a result of chemical alteration by means of the preservative benzalkonium chloride which acts as detergent and changes the epithelial integrity of the Zonulae occludentes, a permeation increase which increases continuously over the test time can be demonstrated. This indicates cumulative and extensive damage which is caused by benzalkonium chloride in long-term application.

Fluorescein Permeation: Ursapharm a (Lecithin Gel+IPM 0.4%; 3 Sets of Data (Experiment 4), FIG. 2)

In FIG. 2, the established substance quantity of fluorescein within the simulated eye anterior chamber is plotted over time after the first application of the test substance Ursapharm A (lecithin gel+IPM 0.4%). The initial value at the time zero was measured directly before the first application. Subsequently, a further value was determined every 24 hours. If between the simulation of the blink (by applying culture medium in drops) in addition the test substance Ursapharm A (lecithin gel+IPM 0.4%) is applied to the cornea, then a significant increase in permeation of fluorescein can be observed over time. This increase is already significant after 24 hours. In the application interval between 48 and 72 hours after the first application, the increase is steepest. This corresponds to the observed behaviour of the known permeation enhancer benzalkonium chloride. The relative increase in the fluorescein permeation within the observation time of 3 days is in the range of 280 to 450 percent and is hence higher than the observed value for benzalkonium chloride (0.001%). This result shows massive damage to the epithelial integrity which is evident already in the macroscopic and also histological drawing. The loss of wing cells and the practically exposed basal cell layer offer no protection from the penetrating dye. Hence the epithelium should be regarded as damaged.

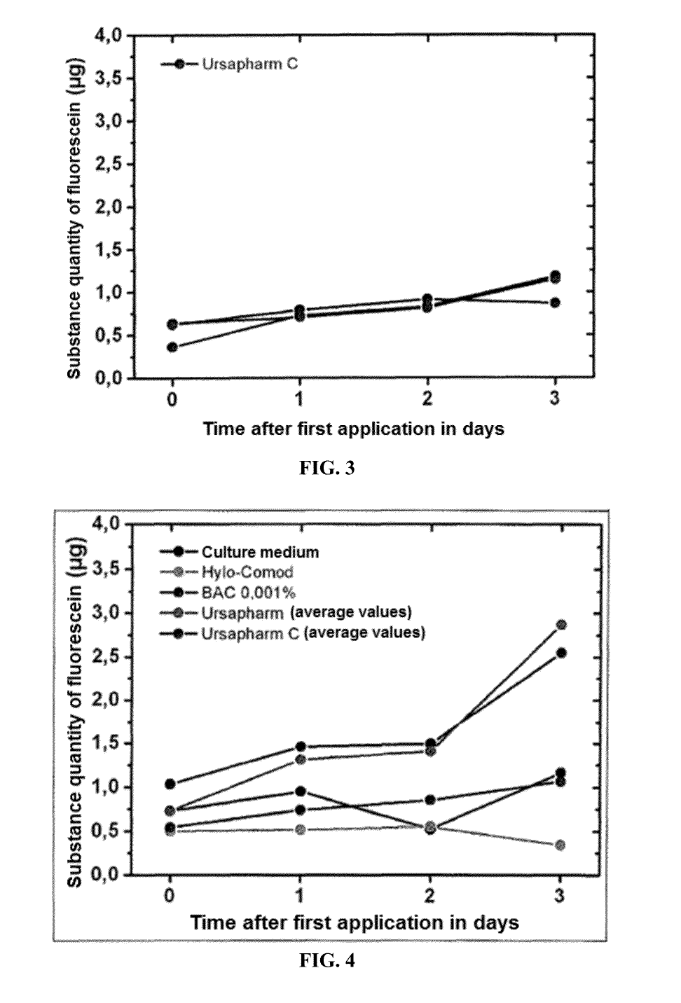

Fluorescein permeation: Ursapharm C (3 sets of data (Experiment 5), FIG. 3)

In FIG. 3, the established substance quantity of fluorescein within the simulated eye anterior chamber is plotted over time after the first application of the test substance Ursapharm C. The initial value at the time zero was measured directly before the first application. Subsequently, a further value was determined every 24 hours. If between the simulation of the blink (due to applying culture medium in drops) in addition the test substance Ursapharm C is applied to the cornea, then a significant but small increase in permeation of fluorescein can be observed over time. This increase corresponds in its entirety to the increase which is likewise observed under normal culture conditions without application of a test substance. The epithelial integrity appears undamaged. The hyperplasia of the anterior epithelial complexes, seen in the histological picture, have no negative influence on the integrity of the Zonulae occludentes which reliably prevent permeation of the dye.

Comparison of the Data (FIG. 4)

FIG. 4 represents a compilation of the above-cited permeation data. For a better overview, respectively average values of the measured substance quantities are indicated here for the permeation data of the test substances Ursapharm A and C which were determined three times. It is detectable that, by means of the vehicle system (Ursapharm A) according to the invention, improved permeation of fluorescein can be achieved compared to all tested compositions.

III. Analysis of the Epithelium Regeneration after Repeated Application of Ursapharm a (Lecithin Gel+IPM 0.4%)

Subsequent to the corneal culture with repeated application of Ursapharm A (lecithin gel+IPM 0.4%, Experiment 4), this corneal culture was cultivated for a further 2 days under standard culture conditions. In addition, 72 hours after the first application of the test substance Ursapharm A (lecithin gel+IPM 0.4%), the culture was continued with culture medium only under hourly simulation of the blink. The cornea was tested 48 hours after the last application of Ursapharm A (lecithin gel+IPM 0.4%), once again by means of OCT and macroscopy. Likewise, a further value for the fluorescein permeation was determined at this time.

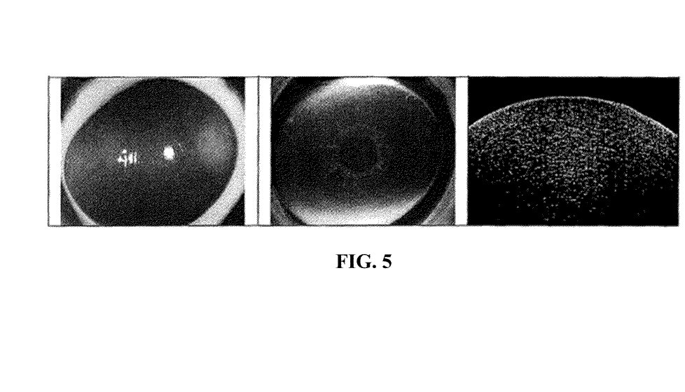

Result: the result of this experiment is illustrated in FIG. 5. From left to right: macroscopy with incident light, macroscopy with transmitted light, optical coherence tomography. It can be detected clearly that the epithelium, starting from the limbal stem cells, is regenerated almost completely within 48 hours without application of Ursapharm A (lecithin gel+IPM 0.4%).

IV. Discussion:

In long-term tests, based on experiments with vital corneal cultures in the EVEIT-long-term system, as expected excellent epithelium integrity is observed with repeated application of HYLO-COMOD®. This is confirmed in the measured values of the fluorescein permeation through the corneas subjected to HYLO-COMOD® drops. The application of HYLO-COMOD® here leads to a constant low permeability over the entire culture time of 3 days. In the case of standard culture conditions with a simulation of the blink by means of application of culture medium, a small increase in permeability is observed during the culture time. From this comparison, a protective effect of HYLO-COMOD® on the Zonula occludens (tight junctions) of the corneal epithelium can be deduced. Histologically, both the corneas cultivated according to standard culture conditions and corneas with HYLO-COMOD® application correspond morphologically to a completely intact cornea. Hence these two culture conditions both correspond to the expected behaviour of a negative control. A similar behaviour is observed for the test substance Ursapharm C. Corneas on which the test substance Ursapharm C was applied repeatedly likewise show no striking features within the observation time of 3 culture days, both in the optical coherence tomography and macroscopically. The substance Ursapharm C maintains the epithelia outstandingly and hence achieves an equivalent behaviour to the negative control culture medium (Experiment 4) and HYLO-COMOD® (Experiment 5). Also with respect to the corneal thickness, substance C is at least equivalent to the negative controls. Histologically, a number of 4-5 wing cell layers was observed in the two tested cultures which were subjected to drops of Ursapharm C. Normally, merely 2, at most 3, cell layers are histologically detectable in the rabbit cornea. In contrast to the negative control HYLO-COMOD®, no protective effect is observed on the Zonula occludens (tight junctions) in the case of Ursapharm C. The fluorescein permeation increases slightly here, comparable to the standard culture conditions, with the culture duration. The corneas suffer a loss of integrity of the epithelia when benzalkonium chloride (0.001%) in Ringer's solution is applied in drops with the solution used as positive control. The same applies to the integrity of the epithelial complex when Ursaphaim A (lecithin gel+IPM 0.4%) is applied in drops. The loss of the epithelial integrity is detectable both in the OCT, because of the reducing epithelial layer thickness and a significantly increasing corneal layer thickness, and by a positive fluorescein detection. This observation is confirmed both in the histological findings and in the very clearly increasing fluorescein permeation through the cornea. The scope of the observed increase in corneal layer thickness confirms the findings of an epithelial disorder and excludes at the same time endothelial disorders which, according to our previous experience with endothelial damage, would accompany a once again substantially higher layer thickness increase. An additional indication as to the loss of epithelial integrity is also the observation that the epithelial layer thickness on the third day after the first application of Ursapharm A (lecithin gel+IPM 0.4%) under moderate evaporation stress (relative air moisture 30% instead of the culture conditions >95%) is reduced within minutes. Such behaviour cannot be observed with negative controls. This may be caused by the detergent effect of lecithin.

After application of the composition according to the invention (Experiment 4), it was shown surprisingly in tests with excised corneas that the barrier function of the cornea is reduced in a reversible manner. When the composition according to the invention is applied in drops, the integrity of the epithelial complex is reduced. This causes an effective active substance permeation through the cornea. This was verified in the tests by means of the model substance fluorescein which is used as model for predominantly paracellularly resorbed active substances. In contrast to the application in drops of the positive control HYLO-COMOD®, during treatment with the composition according to the invention a very significant increase in the fluorescein permeation through the cornea was measured. Surprisingly, it was shown that rapid “rehealing” and proliferation of the epithelial cells is effected.

The application of lecithin as biological detergent likewise has serious consequences, just as benzalkonium chloride. In contrast thereto, this substance does not however impede the proliferation of the epithelia in the “rehealing”. This may be used as an advantage in cases of drugs where an increase in permeation is intended to be achieved without causing the toxic damage of benzalkonium chloride. This result is in stark contrast to the treatment with benzalkonium chloride (0.001%) (Experiment 3) in the tests, in the case of which in fact likewise permeation of fluorescein could be observed. “Rehealing” of the epithelial cells is not effected in this case. The cells were irreversibly damaged; at the same time, the level of fluorescein permeation with the compositions according to the invention is not achieved.

This composition according to the invention hence has a clinically very relevant advantage: it causes a temporary penetration increase for improved absorption of active substance in the eye interior without causing the toxic damage of benzalkonium chloride.

V. Test on the Refractive Index of the Composition According to the Invention

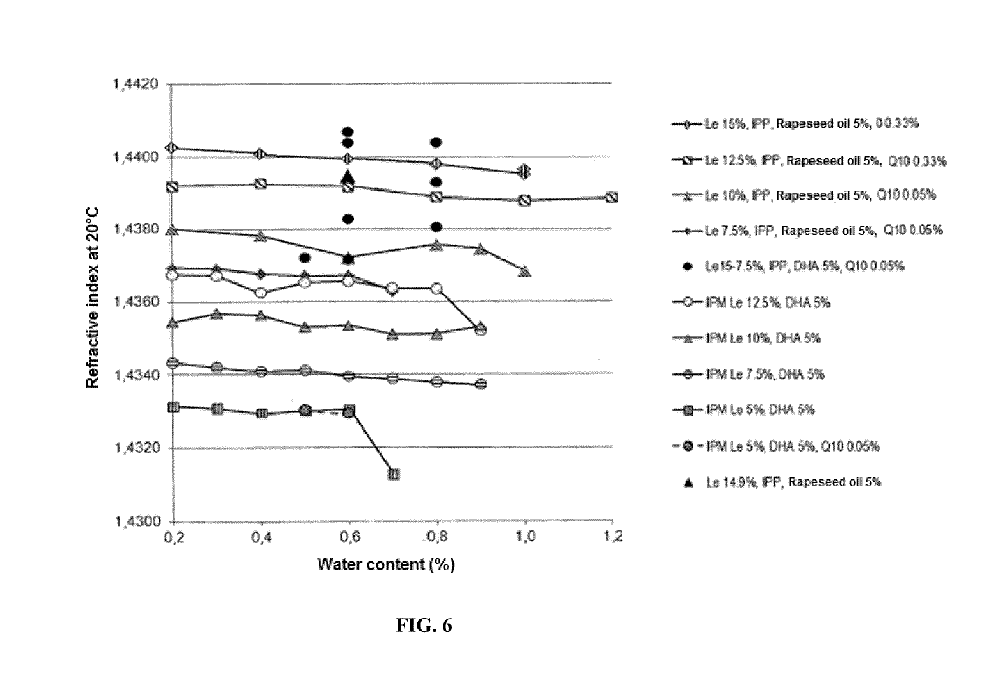

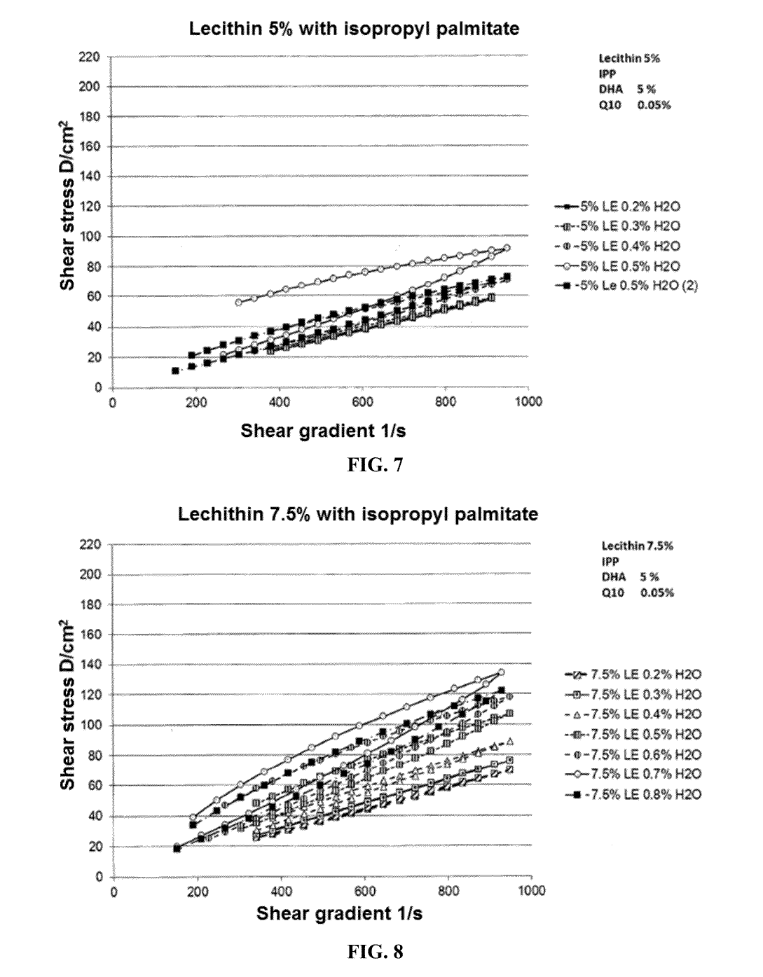

Tests on the refractive indices (at 35° C.) of vehicle systems according to the invention, given by way of example, resulted in these being within values which are suitable for the ophthalmological application field. In FIG. 6, the test results are recorded (Le=lecithin, DHA=docosahexaenoic acid). All formulations thereby have refractive indices in the range between 1.43 and 1.442.

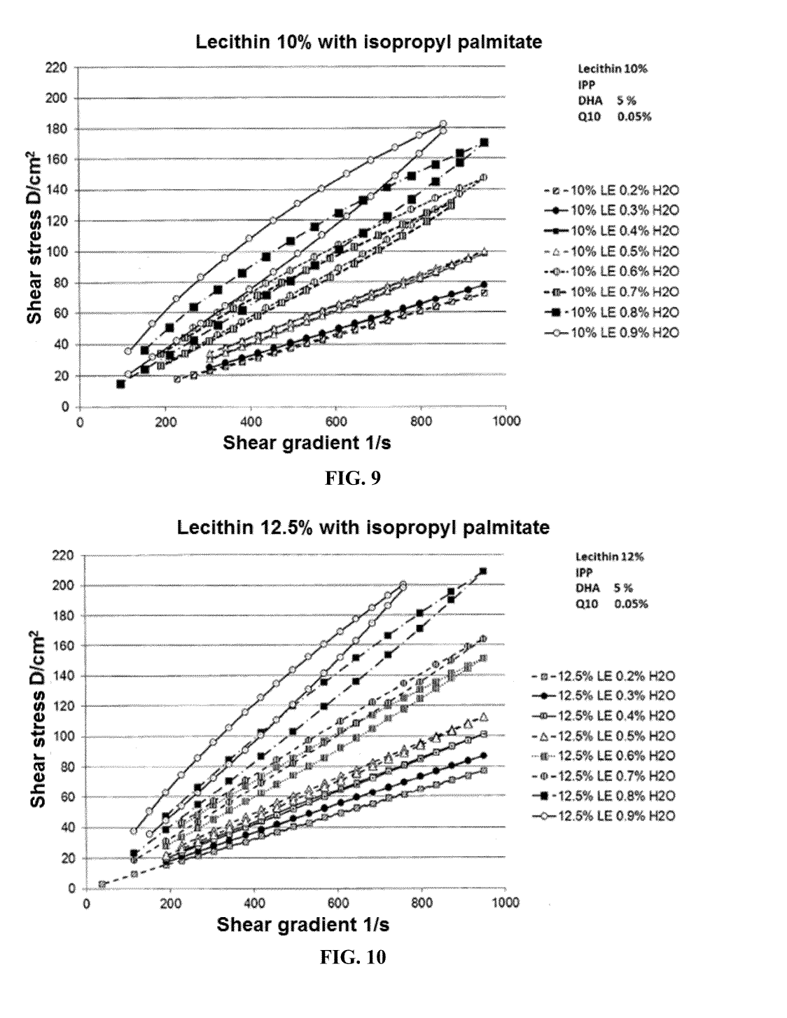

VI. Rheometric Tests on Various Vehicle Systems (FIGS. 8 to 10)

The flow behaviour of ophthalmic formulations, above all of gels and ointments, plays an important role in the perception or feeling of foreign bodies, of the formulation in the eye.

There is understood by thixotropy, both reduction in viscosity with shearing in the course of time, and also the increase in viscosity in the course of time if the sample is no longer sheared or only a little.

The rheological measurements (at 35° C.) verify the thrixotropic properties of vehicle systems according to the invention, given by way of example, and therefore show their suitability for the ophthalmological application field.

Illustrations 8 to 10 show the flow curves for the tested formulations (lecithin with 7.5% by weight, 10% by weight or 12.5% by weight of isopropyl palmitate). The flow curves characterise the flow behaviour at different shear rates. Shear forces/shear rates occur in the eye during blinking. The course of the shear stress i is illustrated as a function of the shear rate. The hysteresis area spanned between the measured curves of the shear rate which is increased over one region and then decelerated again gradually can be seen clearly. The curve, as can be seen in the illustration, does not return on itself. Under shear stress, the viscosity therefore reduces with time. Therefore the forward and return curves are not identical. After experiencing the shear stress, the initial viscosity is built up again. In simplified term's, this means that the thixotropic liquid becomes more and more low-viscous with the duration of its deformation. At the end of the shear stress, the viscosity increases again as a function of time.

This property is desirable for eye formulations, since during blinking, the viscosity of the formulation reduces, consequently is distributed more easily by the eyelid over the eye surface and is not perceived as a foreign body in the eye.

VII. Vitality Determination on Corneal Epithelial Cells in Culture after Application of Various Samples

The background of the test was to test different formulations, which had shown excellent gel stability and consistency, for cytotoxicity.

The cytotoxicity tests were performed on the human corneal epithelial cell line HCE-T, i.e. in the cell test. Cultivation was effected in a black 96 well plate (Nunc). Per well, 25,000 cells were sown and cultivated for 48 h. Determination of the viability was implemented with the Cell Titer Blue® test. The dye resazurin is hereby added to the cells after exposure with the samples. Viable cells are able, because of their mitochondrial activity, to convert the resazurin into the fluorescent resorufin which can be determined fluorimetrically (Illustration 1). Via positive control (Krebs-Ringer-buffer-KRB and saline phosphate buffer—PBS) and also negative control (lysis buffer), the viability of the cells can be expressed as a percentage. All samples were determined with n=6. For the determination, the cell culture medium was removed, the cells were rinsed with KRB and incubated for 60 minutes with the various samples and also controls. Thereafter, the liquids were removed and rinsed three times with KRB plus 1% polysorbate 80 in order to remove residues of the samples. Thereafter, all samples were treated with KRB and resazurin according to Promega protocol and measured with a Genios Plate Reader (Tecan).

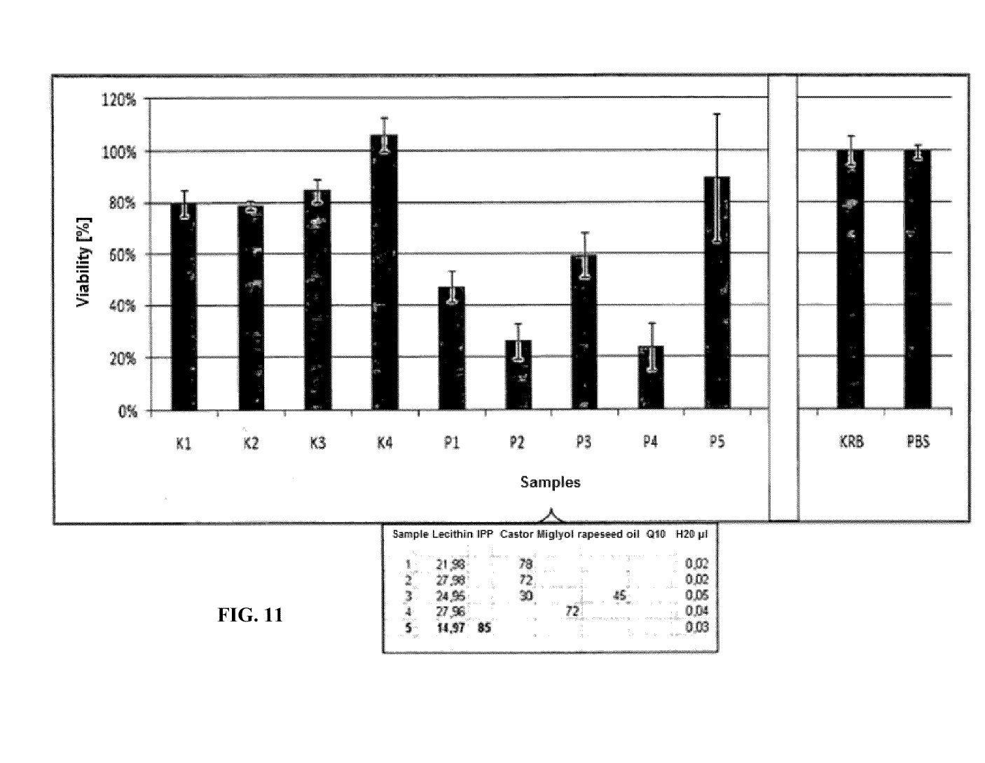

In FIG. 11, test results for vitality determination on corneal epithelial cells in culture are illustrated. For samples K1-4, a viability of 80-100% was shown and, for samples P1-6, generally a significantly reduced viability in the range of 25-90%. Samples P2, 4 and 6 showed in comparison the lowest values for viability. Samples P1 and 3 showed values of around 50% and, for sample P5, a value around 90% could be detected, even though higher values for the standard deviation were determined for the last-mentioned sample.

Comparison of the Controls K1 to K4:

The various gel bases, i.e. apolar phases castor oil (K1), miglyol (K2), rapeseed oil (K3) and isopropyl palmitate (IPP, K4) were tested for cytotoxicity. IPP has the lowest cytotoxicity in the cell test and hence the best tolerability. After treatment with this base, 100% of the cells survive, in contrast to a survival rate of approx. 80% after treatment with all other tested bases.

Comparison of the Samples:

Samples P1 to P5 were optimised with respect to maximum gel stability and consistency. Consequently, in addition to the apolar base, also different lecithin concentrations are chosen.

The comparison of P1 and P2 shows that a lower lecithin concentration with the same base (castor oil) exerts a lower cytotoxic effect. The lecithin concentrations which were selected because of excellent gel stability show, in the cytotoxicity test, too poor tolerability. The lecithin concentrations must be reduced even further for good tolerability. Also sample P3 confirms this trend. Sample P4, with a comparable lecithin concentration to P2, but having the base miglyol, shows a similar cytotoxicity of approx. 75%.

Sample 5, which is based in fact on the most tolerable base IPP and, at approx. 15%, has the lowest concentration of lecithin of the tested samples, shows the best tolerability with a survival rate of 100% of the cells.

These test results in the cell test with human, corneal epithelial cells indicate that a lecithin concentration of less than/equal to 15% is advantageous for the tolerability of the formulation. The cell model represents a very sensitive test system.

1-18. (canceled)

19. An ophthalmological composition comprising
a) ≧30 to 99.95% by weight, relative to the total composition, of at least one fatty acid ester,
b) 0.001% by weight to ≦50% by weight, relative to the total composition, of one or at least one emulsifier, and
c) at least one ophthalmological active substance selected from the group consisting of antibiotics, corticoids, local anaesthetics, decongestants, non-steroidal antiphlogistics, virustatics, antiseptics, cortisone, anti-allergic active substances, prostaglandin analogues, antihistamines, corticosteroids, anti-allergic active substances, pantothenic acid derivatives, non-steroidal anti-inflammatory drugs, vascoconstrictors and anti-glaucoma active substances in a pharmaceutically effective concentration.

20. The ophthalmological composition according to claim 19, wherein
a) the fatty acid ester is selected from the group consisting of isopropyl myristate and isopropyl palmitate and/or
b) the emulsifier is selected from the group of
lecithins;
emulsifiers with HLB values of 2-7;
and mixtures thereof.

21. The ophthalmological composition according to claim 19, wherein
a) the total content of the at least one fatty acid ester, relative to the total composition, is from 50 to 99.9% by weight and/or
b) the total content of the one or at least one emulsifier, relative to the total composition, is from 0.05 to 15% by weight.

22. The ophthalmological composition according to claim 19, comprising at least one ω-3 fatty acid and/or one ω-3 fatty acid derivative selected from the group consisting of esters, mono-, di- or trigylcerides, lipids, oxygenation products, carboxylate salts, amides, other pharmacologically acceptable carboxylic acid derivatives and mixtures thereof.

23. The ophthalmological composition according to claim 22, wherein the at least one ω-3 fatty acid is selected from the group consisting of α-linolenic acid, stearidonic acid, eicosatetraenoic acid, eicosapentaenoic acid (EPA), docosapentaenioc acid (DPA), docosahexaenoic acid, resolvins, hexadecatrienoic acid, eicosatrienoic acid, heneicosapentaenoic acid, tetracosapentaenoic acid, tetracosahexaenoic acid, oxygenation products derived therefrom, and mixtures and combinations hereof.

24. The ophthalmological composition according to claim 22, wherein the at least one ω-3 fatty acid is contained in the form
a) of an ester of an organic alcohol, and/or
b) of a plant or animal oil which comprises, in addition to the at least one ω-3 fatty acid, also at least one ω-6 fatty acid, the molar ratio of the ω-3 fatty acid to the ω-6 fatty acid being from 100:1 to 1:100.

25. The ophthalmological composition according to claim 22, wherein the at least one ω-3 fatty acid is present in the form of an oil, selected from the group consisting of algal oil, fish oil, perilla oil, shi oil, linseed oil, cameline oil, Sacha Inchi oil, rapeseed oil, olive oil, evening primrose oil, soya oil, hemp oil, walnut oil, peanut oil, sesame oil, maize oil, flaxseed oil and mixtures thereof.

26. The ophthalmological composition according to claim 22, wherein the content of the at least one ω-3 fatty acid and/or of the derivative hereof, relative to the total composition, is between 0.01 and 60% by weight.

27. The ophthalmological composition according to claim 19, wherein the composition is free of compounds, selected from the group consisting of quaternary ammonium compounds; sodium glycocholate, and sodium fusidate.

28. The ophthalmological composition according to claim 19, comprising at least one further component, selected from the group consisting of
a) at least one anti-inflammatory- and/or anti-oxidatively- and/or anti-allergically-acting substance, selected from the group consisting of flavonoids, isoflavonoids, polyphenols, anthocyanes, triterpenes, monoterpene alcohols, phenolcarboxylic acids, carotenoids, retinoids, tocopherols (vitamin E), biotin, vitamins A, C, D, K, coenzyme Q (=Q10), carnitine, N-Acetyl-carnitine, glutathione, carnesol, ubiquinone, taurine, plant single substances, substance mixtures, a liquid or solid extract, a distillate or an oil or etheric oil,
b) at least one gel former, selected from the group consisting of natural or synthetic polymers,
c) at least one thickener,
d) at least one moisture-retaining means,
e) at least one auxiliary agent, selected from the group consisting of inorganic buffer substances, organic buffer substances, inorganic salts, organic salts, viscosity regulators, solvents, solubility promoters, solution accelerators, salt formers, viscosity- and consistency controllers, solubilisers, wetting agents, extenders, filling and carrier substances, osmolarity regulators and mixtures thereof, and
f) combinations of the previously mentioned components.

29. The ophthalmological composition according to claim 19, in liquid, viscous or semi-solid form.

30. The ophthalmological composition according to claim 19, wherein said composition is preservative-free.

31. A method for providing prophylaxis and/or treatment of a disease or condition in a patient selected from inflammation, neuritis, cataract, glaucoma, retinopathy, macular degeneration (AMD), retinal detachment, retinoblastoma and choroid melanoma and/or for providing prophylaxis during pre- and/or post-treatment of surgical operations on the eye, cataract extraction with lens implants, refractive-surgical operations, operations on the cornea and corneal transplants or operations on the sclera, said method comprising administering the ophthalmic composition of claim 19 to said patient.

32. The method of claim 31, where the ophthalmic composition is applied topically to the eye, by depositing into the conjunctival sac, or as an insert.

33. The method according to claim 32, wherein said application is effected once daily to once hourly.

34. An ophthalmological kit, comprising
a) an ophthalmological system comprising ≧30 to 99.5% by weight, relative to the total ophthalmological kit, of at least one fatty acid ester and 0.001% by weight to ≦50% by weight, relative to the total ophthalmological kit, of one or at least one emulsifier and
b) at least one ophthalmological active substance selected from the group consisting of antibiotics, corticoids, local anaesthetics, decongestants, non-steroidal antiphlogistics, virustatics, antiseptics, cortisone, anti-allergic active substances, prostaglandin analogues, antihistamines, corticosteroids, anti-allergic active substances, pantothenic acid derivatives, non-steroidal anti-inflammatory drugs, vascoconstrictors and anti-glaucoma active substances in a pharmaceutically effective concentration,
as separate formulations.

35. A dispenser for a sterile fluid, having
a) a passage which connects an inlet opening for a fluid contained in a storage container made of a flexible material and an outflow opening for dispensing the fluid and has therein at least one oligodynamically active substance which is in contact with the fluid;
b) a metering pump which operates without compressed air compensation, comprising an inlet valve for closing the inlet opening, the inlet valve having a material which can interact with the fluid via an oligodynamically active substance; and
c) a spring mechanism which can be in contact with the fluid, the inlet valve and the spring mechanism having a stainless steel material as an oligodynamically active substance and a decontamination mechanism being provided in the upper part of the outlet channel, the decontamination mechanism having a material which can interact with the fluid via an oligodynamic substance which is selected from the group consisting of silver, silver salts, other silver compounds, alloys and nanomers thereof in either metallic or salt form or as a chemical compound thereof, wherein the fluid contained in the storage container is an ophthalmological composition according to claim 19.

36. The ophthalmologic composition of claim 20, wherein the emulsifiers with HLB values of 2-7 are selected from ethoxylated triglycerides, sorbitan esters, polyethoxylated fatty acids, and mixtures thereof.
